SEQUENCE LISTING (13 sequences)

SEQ ID NO: 1 - protein, 5 aa, Homo sapiens
YGGFM

SEQ ID NO: 2 - protein, 5 aa, Homo sapiens
YGGFL

SEQ ID NO: 3 - protein, 6 aa, Artificial Sequence
  Homo sapiens-synthetic peptide
YGGFLR

SEQ ID NO: 4 - protein, 6 aa, Artificial Sequence
  Homo sapiens-synthetic peptide
YGGFMR

SEQ ID NO: 5 - protein, 6 aa, Artificial Sequence
  Homo sapiens-synthetic peptide
YGGFMK

SEQ ID NO: 6 - protein, 7 aa, Artificial Sequence
  Homo sapiens-synthetic peptide
YGGFMRR

SEQ ID NO: 7 - protein, 7 aa, Artificial Sequence
  Homo sapiens-synthetic peptide
YGGFMRF

SEQ ID NO: 8 - protein, 8 aa, Artificial Sequence
  Homo sapiens-synthetic peptide
YGGFMRGL

SEQ ID NO: 9 - protein, 10 aa, Artificial Sequence
  Homo sapiens-synthetic peptide
YGGFLRKYPK

SEQ ID NO: 10 - protein, 9 aa, Artificial Sequence
  Homo sapiens-synthetic peptide
YGGFLRKYP

SEQ ID NO: 11 - protein, 8 aa, Artificial Sequence
  Homo sapiens-synthetic peptide
YGGFLRRI

SEQ ID NO: 12 - protein, 31 aa, Homo sapiens
YGGFMTSEKSQTPLVTLFKNAIIKNAYKKGE

SEQ ID NO: 13 - protein, 17 aa, Homo sapiens
YGGFLRRIRPKLKWDNQ

(19) United States
(12) Patent Application Publication
(10) Pub. No.: US 2007/0142287 A1
(43) Pub. Date: Jun. 21, 2007

(54) Compositions And Methods For Treatment Of Cancer

(75) Inventor: Farhan Taghizadeh, Albuquerque, NM (US)

Correspondence Address: PROTEUS PATENT PRACTICE LLC, P.O. BOX 1867, NEW HAVEN, CT, 06508, US

(73) Assignee: Biomed Solutions, LLC, West Henrietta, NY (US)

(21) Appl. No.: 11/613,664

(22) Filed: Dec. 20, 2006

Related U.S. Application Data
(60) Provisional application No. 60/752,426, filed on Dec. 20, 2005.

(51) Int. Cl. A61K 38/33

(52) U.S. Cl. 514/12

(57) ABSTRACT

The present invention provides a pharmaceutical composition that includes a) one or both of i) a peptide ligand that binds a zeta receptor and ii) at least one cancer chemotherapeutic agent; and b) a sustained release formulation. The invention also provides a combination including at least a portion of a tissue comprising one or more cells harboring a zeta receptor, such as a cancer cell or a precancerous cell, in contact with a pharmaceutical composition of the invention. Additionally the invention provides a method of treating a pathology that responds to a peptide ligand that binds a zeta receptor that includes a step of contacting a tissue characteristic of the pathology and containing cells harboring a zeta receptor, with a pharmaceutical composition of the invention. The invention further provides methods of inhibiting proliferation or metastasis of a cancer in a subject, including a) surgically excising a tumor characterizing the cancer from the subject; b) ensuring maximal removal of a tumor margin from the tumor site; and c) disposing a pharmaceutical composition of the invention upon a tissue remaining at a tumor margin wherein the tissue contains one or more tumor cells (such as a cancer cell or a precancerous cell) harboring a zeta receptor. The peptide ligand of a zeta receptor may be an enkephalin, an endorphin, a dynorphin, or a derivative thereof. Additionally the pharmaceutical composition may include one or more cancer chemotherapeutic agents.

### REFERENCE TO SEQUENCE LISTING

The present invention includes a sequence listing summarized in Table 1.

TABLE 1

| Description | SEQ ID NO: |
|---|---|
| Met⁵-Enkephalin | 1 |
| Leu⁵-Enkephalin | 2 |
| Arg⁶-Leu-Enkephalin | 3 |
| Arg⁶-Met-Enkephalin | 4 |
| Lys⁶-Met-Enkephalin | 5 |
| Arg⁶-Arg⁷-Met-Enkephalin | 6 |
| Arg⁶-Phe⁷-Met-Enkephalin | 7 |
| Arg⁶-Gly⁷-Leu⁸-Met-Enkephalin | 8 |
| Alpha-Neo-Endorphin | 9 |
| Beta-Neo-Endorphin | 10 |
| Ph-8p (Dynorphin [1-8]) | 11 |
| Human Endorphin | 12 |
| Human Dynorphin | 13 |

### BACKGROUND OF THE INVENTION

Cancers of various organs and tissues is a major cause of illness and death among the population. In many cases a significant aspect in the treatment of cancer is resection or ablation of the malignant tumor. The surgeon's objective is to remove as much of the tumor as possible, while leaving healthy, or normal, tissue intact in situ.

Certain problems remain with this approach. It is difficult, in the real time during which the surgery proceeds, effectively to identify and remove cancerous cells and tissue from the tumor margin. In addition, as a result of the surgical manipulations, cancer cells may be abraded or dislodged from the mass of the tumor and remain within the cavity opened by the surgery. These two potential sources of cancer cells may then remain behind within the body of the patient. As such, they represent potential sources for recurrence of the malignancy after the passage of time.

In current practice, this potential source of recurrence of the malignancy is addressed, for example, by secondary treatments such as systemic administration of anti-cancer chemotherapeutic agents, or by radiation treatment, or a combination thereof. Systemic administration is widely known to have additional problems, including secondary effects or side effects within the body of a patient, leading to discomfort and distress for the patient.

In recent years the ability of endorphins, such as Met⁵-enkephalin, to bind the 220 kD receptor nuclear protein termed the zeta opioid receptor and inhibit cell growth has been well established. This receptor is found in many cells, both within the central nervous system and in other tissues and organs. The ability of exogenously provided endorphins to bind the zeta receptor and retard tumor growth has also been well characterized. There seems to be a higher affinity for Met⁵-enkephalin to the zeta receptor in one cell line tested, though this selectivity does not necessarily mean exclusivity, and many other opioids and endorphins bind this receptor. Similarly, opioid receptor antagonists like naloxone and naltrexone have been shown to increase cell growth by blocking this receptor.

The translation of these in vitro and in vivo studies on the properties of the Met⁵-enkephalin-zeta receptor system into effective clinical therapy for the treatment of cancer presents many challenges. The primary challenge is how initial tumor burden impacts the final outcome of this drug given systemically. Most studies to date have focused on the infusion of the drug systemically, ranging from subcutaneous injections, to intraperitoneal injections, to daily and continuous parenteral infusions. These studies are predicated on use of Met⁵-enkephalin as the sole treatment modality for the aberrantly dividing tumor cells. Even Phase 1 trials for using specifically Met⁵-enkephalin in humans with pancreatic cancer focused only on systemic treatment of end stage patients who had unresectable tumors. Certain limited experiments have been completed on intratumoral injection of Met⁵-enkephalin (Zagon I S, Jaglowski J R, Verderame M F, Smith J P, Leure-Dupree A E, McLaughlin P J. Cancer Chemother Pharmacol. 2005 November; 56(5):510-20. Epub 2005 Jun. 10. Combination chemotherapy with gemcitabine and biotherapy with opioid growth factor (OGF) enhances the growth inhibition of pancreatic adenocarcinoma; McLaughlin P J, Levin R J, Zagon I S. Opioid growth factor (OGF) inhibits the progression of human squamous cell carcinoma of the head and neck transplanted into nude mice. Cancer Lett. 2003 Sep. 25; 199(2):209-17), according to which the short exposure time of the drug and the apparently unreproducible process of injecting a drug directly into an intact tumor gave inconsistent results. The authors of that experiment noted that a longer time of OGF (i.e., Met⁵-enkephalin) exposure by the intratumoral route might yield different results. Those authors failed to indicate how this might be carried out, however.

There thus remain important deficiencies in therapeutic approaches to the treatment of various cancers. For example, there remains a strong need to inhibit recrudescence of a tumor at the site of a surgical resection, cryosurgical removal, or ablation. There additionally remains a need to interfere with growth of cancerous or precancerous cells remaining at the margin of a tumor after removal of the bulk of the tumor mass. There further remains a need to cause cancerous or precancerous cells remaining at the margin of a tumor to die, or to kill them. There also remains a need to provide anti-cancer chemotherapeutic agents over a sustained time frame locally at a tumor margin. The present invention addresses these and related needs and deficiencies in the field of oncology and treatment of various cancers.

### BRIEF SUMMARY OF THE INVENTION

In a first aspect the present invention provides a composition, pharmaceutical composition, or medicament that includes a) one or both of i) a peptide ligand that binds a zeta receptor and ii) at least one cancer chemotherapeutic agent; and b) a sustained release formulation suitable for topical application to a tissue.

In a further aspect the invention provides a method of preparing a pharmaceutical composition that includes combining a) one or both of i) a peptide ligand that binds a zeta receptor and ii) at least one cancer chemotherapeutic agent; and b) a sustained release formulation suitable for topical application to a tissue.

In an additional aspect the present invention provides a combination including at least a portion of a tissue comprising one or more cells harboring a zeta receptor, in contact with a pharmaceutical composition that includes

a) one or both of
i) a peptide ligand that binds a zeta receptor and
ii) at least one cancer chemotherapeutic agent, and

b) a sustained release formulation suitable for topical application to the tissue.

In still an additional aspect the present invention provides a method of forming a combination that includes a step of disposing a pharmaceutical composition upon at least a portion of a tissue that contains one or more cells harboring a zeta receptor, wherein the pharmaceutical composition contains

a) one or both of
i) a peptide ligand that binds a zeta receptor and
ii) at least one cancer chemotherapeutic agent; and

b) a sustained release formulation suitable for topical application to the tissue.
In these combinations, and in combinations resulting from the disposing step in methods of forming a combination, the pharmaceutical composition and at least the portion of the tissue having cells harboring a zeta receptor are physically juxtaposed with respect to each other. In various embodiments of combinations, and in methods of forming a combination, the one or more cells are a cancer cell or a precancerous cell. In various embodiments of combinations, and in methods of forming combinations, the pharmaceutical composition is in contact with, or is disposed upon, a tumor margin, or the pharmaceutical composition is applied to a tumor margin, wherein the margin is exposed by removal of a preponderance of the tumor. In further embodiments of combinations, and methods of forming combinations, the one or more cells are located in a subject.

In various embodiments of compositions, methods of preparing compositions, combinations, and methods of forming combinations of this invention, the composition includes a peptide ligand of a zeta receptor, which may be an enkephalin, a fragment of an enkephalin, or a derivative thereof; or a zeta receptor peptide ligand may be an endorphin, a fragment of an endorphin, or a derivative thereof, or a zeta receptor peptide ligand may be a dynorphin, a fragment of a dynorphin, or a derivative thereof. A fragment or a derivative of an enkephalin, and endorphin, or a dynorphin exhibits at least one biological activity of the parent enkephalin, endorphin, or dynorphin. In yet additional embodiments the compositions and combinations, and the methods, include one or more cancer chemotherapeutic agents, such as, by way of nonlimiting example, the cancer chemotherapeutic agents disclosed herein.

In still additional embodiments of compositions, methods of preparing compositions, combinations, and methods of forming combinations, the composition includes both a peptide ligand of a zeta receptor and at least one cancer chemotherapeutic agent. The peptide component may be an enkephalin, a fragment of an enkephalin, or a derivative thereof, or an endorphin, a fragment of an endorphin, or a derivative thereof; or a dynorphin, a fragment of a dynorphin, or a derivative thereof.

The sustained release formulation included or employed in the compositions, combinations, and methods of the invention may be any of a wide range of formulations. In general, sustained release formulations employed in various embodiments of the compositions, combinations and methods of the present invention are biocompatible, and in further embodiments they are biodegradable. Nonlimiting examples of such formulations include gels, hydrogels, crosslinked gels, formulations applied by spray, bioadhesive formulations, and similar formulations, or a combination of any of them. Additionally, a sustained release composition, or a combination, may be constituted of, or may include as one of several components, and methods of forming combinations may employ compositions that include, particles, beads, microparticles, nanoparticles, and similar formed entities that include a pharmaceutically active agent as a component.

Furthermore, compositions or combinations that include such compositions, and methods of the invention, may contain or employ a peptide ligand that binds a zeta receptor and/or at least one cancer chemotherapeutic agent, or a precursor thereof that gives rise to the pharmaceutically active agent in situ, conjugated to a polymer component of a sustained release formulation. In general, a composition, or a composition included in a combination releases the pharmaceutically active agent in an amount substantially effective to treat a cancer cell over a period of time that may endure 1 day or longer, and may be as long as 13 weeks, or even longer.

In yet an additional aspect the present invention provides a method of treating a pathology that responds to a peptide ligand that binds a zeta receptor. The method includes a step of disposing a pharmaceutical composition upon at least a portion of a tissue suspected of comprising one or more cells exhibiting the pathology wherein the one or more cells harbor a zeta receptor, wherein the pharmaceutical composition includes

a) one or both of
i) a peptide ligand that binds a zeta receptor and
ii) at least one cancer chemotherapeutic agent, in amounts effective to treat the pathology; and

b) a sustained release formulation suitable for topical application to a tissue.

In various embodiments of this method the pathology is a cancer, and in further various embodiments of this method the one or more cells is a cancer cell or a precancerous cell. Additionally in various embodiments of methods of treating a pathology the one or more cells are located in a subject. In various additional embodiments of methods of treating a pathology the one or more cells are at a margin of a tumor wherein the margin is exposed by removal of a preponderance of the tumor. In various embodiments of methods of treating a pathology the medicament is applied to contact a tumor margin.

In still a further aspect the invention provides various methods of inhibiting proliferation or metastasis of a cancer in a subject. These methods include the steps of
a) surgically excising a tumor characterizing the cancer from the subject;
b) ensuring maximal removal of a tumor margin from the tumor site; and
c) disposing a medicament upon at least a portion of a tissue remaining at a tumor margin wherein the tissue contains one or more tumor cells (such as a cancer cell or a precancerous cell) harboring a zeta receptor. The medicament disposed on the tissue contains
   i) one or both of
      a′) a peptide ligand that binds a zeta receptor and
      b′) at least one cancer chemotherapeutic agent, in amounts effective to treat the tumor cells; and
   ii) a sustained release formulation suitable for topical application to a tissue.
      The steps of this method are effective to inhibit proliferation or metastasis of the cancer.

In various embodiments of methods of treating a pathology and methods of inhibiting proliferation or metastasis, a pharmaceutical composition or medicament employed in the methods includes a) one or both of a peptide ligand that binds a zeta receptor and at least one cancer chemotherapeutic agent; and b) a sustained release formulation. In various additional embodiments of methods of treating a pathology and methods of inhibiting proliferation or metastasis, the pharmaceutical composition or medicament includes a peptide ligand of a zeta receptor that may be an enkephalin, a fragment of an enkephalin, or a derivative thereof, or an endorphin, a fragment of an endorphin, or a derivative thereof; or a dynorphin, a fragment of a dynorphin, or a derivative thereof. A fragment or a derivative of an enkephalin, an endorphin, or a dynorphin exhibits at least one biological activity of the parent enkephalin, endorphin, or dynorphin, respectively. In yet additional embodiments the pharmaceutical composition or medicament includes one or more cancer chemotherapeutic agents.

In still additional embodiments of methods of treating a pathology and methods of inhibiting proliferation or metastasis, the pharmaceutical composition or medicament includes both a peptide ligand of a zeta receptor and at least one cancer chemotherapeutic agent. The peptide component may be an enkephalin, a fragment of an enkephalin, or a derivative thereof; or an endorphin, a fragment of an endorphin, or a derivative thereof; or a dynorphin, a fragment of a dynorphin, or a derivative thereof.

In general, sustained release formulations employed in methods of treating a pathology and methods of inhibiting proliferation or metastasis of the present invention are biocompatible, and in many embodiments are biodegradable. Nonlimiting examples of such formulations include gels, hydrogels, formulations applied by spray, bioadhesive formulations, and similar formulations. Additionally, sustained release formulations employed in methods of treating a pathology and methods of inhibiting proliferation or metastasis may be constituted of, or may include as one of several components, particles, beads, microparticles, nanoparticles, and similar formed entities that include a pharmaceutically active agent as a component, or a combination of any of them. In additional various embodiments of methods of treating a pathology and methods of inhibiting proliferation or metastasis, release of the peptide ligand, or the one or more cancer chemotherapeutic agents, or both, contained therein from the pharmaceutical composition or medicament occurs in a substantially therapeutically effective amount over a time period ranging from about 1 day to about 13 weeks. In yet additional various embodiments the methods of treating a pathology, and the methods of inhibiting proliferation or metastasis, further include systemic administration of a cancer chemotherapeutic agent via a parenteral route.

### BRIEF DESCRIPTION OF THE DRAWINGS

FIG. 1. Schematic representations of various embodiments of pharmaceutical compositions of the invention. (a) Homogenous composition; (b) heterogeneous composition including an active agent not soluble in a sustained release formulation; (c) heterogeneous composition including microparticles that contain an active agent disposed therein.

FIG. 2. Schematic representations of various embodiments of combinations of the invention. (a) Combination of a tissue with a homogenous composition; (b) combination of a tissue with a heterogeneous composition including an active agent not soluble in a sustained release formulation; (c) combination of a tissue with a heterogeneous composition including microparticles that contain an active agent disposed therein.

FIG. 3. Schematic representation of an embodiment of a method of inhibiting proliferation or metastasis of a cancer.

FIG. 4. An embodiment of a procedure for inhibiting proliferation or metastasis of a cancer.

FIG. 5. An embodiment of a procedure for inhibiting disease progression of a precancerous lesion.

### DETAILED DESCRIPTION OF THE INVENTION

The present invention provides compositions and methods directed toward inhibiting reappearance of a tumor after the tumor has been removed by a surgical resection, cryosurgical removal, ablation, or comparable procedure. These compositions and methods lead to inhibition of growth, or actual death, of cancerous or precancerous cells remaining at the margin of a tumor after removal of the bulk of the tumor mass. The compositions and methods of the invention include provision of anti-cancer chemotherapeutic agents over a sustained time frame locally at a tumor margin. Thus the present invention provides valuable and useful improvements in oncology and treatment of various cancers.

As used herein, the term “enkephalin” and similar terms and phrases relate to a pentapeptide produced in vivo by proteolytic processing from precursor polypeptides such as human corticotropin-beta-lipotropin precursor ((CCLP) pro-opiomelanocortin; see, by way of nonlimiting example, GenBank Acc. No. P01189), or from human beta-neoendorphin-dynorphin precursor ((NDP) proenkephalin; see by way of nonlimiting example GenBank Acc. No. P01213). Naturally occurring amino acids and their derivatives are represented by three-letter codes according to IUPAC conventions. When there is no indication, L isomer is intended. D isomers are indicated by “D” before the residue. At least two naturally occurring enkephalins are known:

Tyr-Gly-Gly-Phe-Met (Met⁵-enkephalin;
SEQ ID NO 1:),
and
Tyr-Gly-Gly-Phe-Leu (Leu⁵-enkephalin;
SEQ ID NO 2:).

Met⁵-enkephalin originates from residues 237-241 of human corticotropin-lipotropin precursor, and Leu⁵-enkephalin originates from residues 207-211 of human beta-neoendorphin-dynorphin precursor (see below). The term “enkephalin”, as used herein, encompasses both Met⁵-enkephalin and Leu⁵-enkephalin. In addition, the term “enkephalin” encompasses the following closely related enkephalins and endorphins (Table 2):

TABLE 2

| | | |
|---|---|---|
| | | SEQ |
| | | ID |
| Name | Sequence | NO: |

| | | |
|---|---|---|
| Arg⁶-Leu- | Tyr-Gly-Gly-Phe-Leu-Arg | 3 |
| enkephalin | | |
| Arg⁶-Met- | Tyr-Gly-Gly-Phe-Met-Arg | 4 |
| enkephalin | | |
| Lys⁶-Met- | Tyr-Gly-Gly-Phe-Met-Lys | 5 |
| enkephalin | | |
| Arg⁶-Arg⁷-Met- | Tyr-Gly-Gly-Phe-Met-Arg-Arg | 6 |
| enkephalin | | |
| Arg⁶-Phe⁷-Met- | Tyr-Gly-Gly-Phe-Met-Arg-Phe | 7 |
| enkephalin | | |
| Arg⁶-Gly⁷-Leu⁸- | Tyr-Gly-Gly-Phe-Met-Arg-Gly-Leu | 8 |
| Met-enkephalin | | |
| Alpha-Neo | Tyr-Gly-Gly-Phe-Leu-Arg-Lys-Tyr-Pro-Lys | 9 |
| Endorphin | | |
| Beta-Neo | Tyr-Gly-Gly-Phe-Leu-Arg-Lys-Tyr-Pro | 10 |
| Endorphin | | |
| Ph-8P (Dynorphin | Tyr-Gly-Gly-Phe-Leu-Arg-Arg-Ile | 11 |
| [1-8]) | | |

Human endorphin is a processed product obtained as the C-terminal residues 237-267 of human corticotropin-lipotropin precursor (Pro-opiomelanocortin) (see, for example, GenBank Acc. No. (from Swissprot) P01189). The sequence of human endorphin is given by:

(SEQ ID NO:12)

Tyr Gly Gly Phe Met Thr Ser Glu Lys Ser Gln Thr
Pro Leu Val Thr Leu Phe Lys Asn Ala Ile Ile Lys
Asn Ala Tyr Lys Lys Gly Glu

Human dynorphin is a processed product obtained as an internal peptide given by residues 207-223 of human beta-neoendorphin-dynorphin precursor (Proenkephalin B; Preprodynorphin) (see, for example, GenBank Acc. No. (from Swissprot) P01213). The sequence of human dynorphin is given by:

(SEQ ID NO:13)

Tyr Gly Gly Phe Leu Arg Arg Ile Arg Pro Lys Leu
Lys Trp Asp Asn Gln

A wide variety of derivatives of an enkephalin, an endorphin or a dynorphin is also encompassed within the scope of the invention. In one class of derivative, any one of the amino acid residues in an enkephalin, an endorphin or a dynorphin may be substituted by another of the naturally occurring amino acid residues. In an additional class of derivative, any one or more, or even all, the residues of an enkephalin, an endorphin or a dynorphin or a derivative of an enkephalin, an endorphin or a dynorphin may be a D-amino acid instead of a naturally-occurring L-amino acid. Yet additional derivatives of an enkephalin, an endorphin or a dynorphin include one, two, or more naturally occurring amino acid residues or derivatives of amino acid residues bonded to the N-terminal residue or to the C-terminal residue. Still further derivatives of an enkephalin, an endorphin or a dynorphin include an acyl moiety bonded to the N-terminus (forming a carboxamide derivative), or an amino moiety (forming an amide derivative) or an oxo-organic radical moiety (forming an ester derivative) bonded to the C-terminus. Still additional derivatives of an enkephalin, an endorphin or a dynorphin include fragments of an enkephalin, an endorphin or a dynorphin, respectively. Still further derivatives of an enkephalin, an endorphin or a dynorphin include fragments of an enkephalin, an endorphin or a dynorphin, respectively, that in turn are further derivatized according to the disclosures in the following paragraphs.

Examples of C-terminal or N-terminal modifications of peptides that may be used to prepare derivatives of an enkephalin, an endorphin or a dynorphin are described, for example, in Green and Wuts, “Protecting Groups in Organic Synthesis”, John Wiley and Sons, Chapters 5 and 7, 1991, the teachings of which are incorporated herein by reference. Various modifying groups include N-terminal protecting groups such as acyl groups (—CO—R₁) and alkoxy carbonyl or aryloxy carbonyl groups (—CO—O—R₁), wherein R₁ is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, phenyl-CO—, substituted phenyl-CO—, benzyl-CO— and (substituted benzyl)-CO—. Examples of alkoxy carbonyl and aryloxy carbonyl groups include CH₃—O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—. In additional embodiments of derivatives of an enkephalin, an endorphin or a dynorphin, the carboxyl group at the C-terminus is protected, for example, as an amide (i.e., the hydroxyl group at the C-terminus may be replaced with-NH₂, —NHR₁ and —NR₂R₃) or ester (i.e. the hydroxyl group at the C-terminus may be replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH₃)₂, —NH(ethyl), —N(ethyl)₂, —N(methyl)(ethyl), —NH(benzyl), —N(C1-C4 alkyl)(benzyl), —NH(phenyl), —N(C1-C4 alkyl)(phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

Examples of non-naturally occurring amino acids that may be substituted for a natural amino acid of an enkephalin, endorphin or dynorphin include the following alpha-amino acids: 2-aminobutyric acid, 2-amino-isobutyric acid, beta-alanine, beta-(4-biphenyl)-alanine, citrulline, diaminobutyric acid, homoarginine, homocysteine, 1-naphthylalanine, 2-naphthylalanine, norvaline, p-chlorophenylalanine, p-aminophenylalanine, p-fluorophenylalanine, p-nitrophenylalanine, 4-pyridylalanine. norleucine, alpha,beta-diaminopropionic acid, cyclohexylalanine, norvaline, beta-(3-pyridinyl)alanine, 1-amino-1-cyclo-hexanecarboxylic acid, alpha-aminoisobutyric acid, 1-amino-1-cyclopropanecarboxylic acid, 1-amino-1-cyclobutanecarboxylic acid, 1-amino-1-cyclopentanecarboxylic acid, 1-amino-1-cyclohexanecarboxylic acid, 1-amino-1-cycloheptanecarboxylic acid, 1-amino-1-cyclooctanecarboxylic acid, and 1-amino-1-cyclononanecarboxylic acid. In addition, an alkyl or aryl residue in the preceding amino acids may contain 1-4 hydroxy substituents. Additional examples of amino acyl derivatives that may be incorporated into an enkephalin derivative are disclosed in U.S. Pat. No. 6,841,657, incorporated herewith in its entirety.

By way of nonlimiting example, a derivatized amino acid residue may further have the following side chains: ethyl, n-butyl, —CH₂—CH₂—OH, —CH₂—CH₂—CH₂—OH, —CH₂—CHOHCH₃ and —CH₂—S—CH₃; phenylglycine; cyclohexylmethylalanine; modified amino residues having substituted benzyl or phenyl side chains wherein preferred substituents include one or more of the following: halogen, methyl, ethyl, nitro, methoxy, ethoxy and —CN; a substituted or unsubstituted aliphatic, aromatic or benzylic ester of glutamic or aspartic acid (e.g., methyl, ethyl, n-propyl iso-propyl, cyclohexyl, benzyl or substituted benzyl); CO—NH-alkylated glutamate or asparagine (e.g., methyl, ethyl, n-propyl and iso-propyl); and modified amino acids having the side chain —(CH₂)₃—COOH, an ester thereof (substituted or unsubstituted aliphatic, aromatic or benzylic ester), an amide thereof or a substituted or unsubstituted N-alkylated amide thereof; N-nitroarginine; beta-cycloarginine; gamma-hydroxyarginine; N-amidinocitruline; 2-amino-4-guanidinobutanoic acid; homologs of lysine; homologs of arginine; and modified amino acids having C1-C5 straight or branched alkyl side chains substituted with —OH or —SH.

Provided below are groups of naturally occurring and modified amino acids in which each amino acid in a group has similar electronic and steric properties. Generally, it is believed that a conservative substitution can be made by substituting an amino acid with another amino acid from the same group. It is to be understood that these groups are non-limiting, i.e. that there are additional modified amino acids which could be included in each group.

Group I includes leucine, isoleucine, valine, methionine, serine, cysteine, threonine and modified amino acids having the following side chains: ethyl, n-butyl, —CH₂—CH₂—OH, —CH₂—CH₂—CH₂—OH, —CH₂—CHOH—CH₃ and —CH₂—S—CH₃.

Group II includes glycine, alanine, valine, serine, cysteine, and threonine.

Group III includes phenylalanine, phenylglycine, tyrosine, tryptophan, cyclohexylmethylalanine, and modified amino residues having substituted benzyl or phenyl side chains. Preferred substituents include one or more of the following: halogen, methyl, ethyl, nitro, methoxy, ethoxy and —CN.

Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamate, asparagine, CO—NH-alkylated glutamate or asparagine (e.g., methyl, ethyl, n-propyl and iso-propyl) and modified amino acids having the side chain —(CH₂)₃—COOH, an ester thereof (substituted or unsubstituted aliphatic, aromatic or benzylic ester), an amide thereof and a substituted or unsubstituted N-alkylated amide thereof.

Group V includes histidine, lysine, arginine, N-nitroarginine, beta-cycloarginine, .gamma.-hydroxyarginine, N-amidinocitruline and 2-amino-4-guanidinobutanoic acid, homologs of lysine, homologs of arginine and ornithine. A homolog of an amino acid includes from 1 to about 3 additional methylene units in the side chain.

Group VI includes serine, threonine, cysteine and modified amino acids having C1-C5 straight or branched alkyl side chains substituted with —OH or —SH.

Derivatives or fragments of an enkephalin, an endorphin or a dynorphin such as those disclosed above, and others that are equivalent thereto, and that possess zeta receptor binding properties or cancer therapeutic characteristics as described herein, are encompassed within the scope of the compositions, combinations and methods of the present invention.

As used herein the term “zeta receptor” and similar terms or phrases relate generally to a nuclear receptor which binds opioid substances, including opioid peptides such as enkephalins, endorphins and dynorphins and derivatives thereof; a zeta receptor additionally binds a variety of opioid agonists and opioid antagonists. A zeta receptor occurs in mammalian cells, including human cells. It is a nuclear receptor of about 220 kD. A zeta receptor has also been referred to as “zeta opioid receptor” or “zeta enkephalin receptor”. A particular pentapeptide, Met⁵-enkephalin, has been identified as an endogenous opioid directly involved in growth processes, serving as a negative regulator in a wide variety of cells and tissues. Upon binding an enkephalin or other effective opioid agonist, a zeta receptor signals within the cell in which it occurs to inhibit or retard cell growth, cell proliferation, cell migration, differentiation, and survival. For this reason an enkephalin (specifically, Met⁵-enkephalin) inducing such a response has been termed an “opioid growth factor” (OGF), since it exhibits a function other than as a neurotransmitter, and has been found in tissues other than neurotransmitter cells. An enkephalin that binds to a zeta receptor thus elicits a negative growth response. The zeta receptor is disclosed in U.S. Pat. No. 6,737,397, and is further characterized, for example, in McLaughlin, P J, Int'l J. Oncol. 24: 227-232 (2004); Zagon I S, Am J. Physiol. 1995:R 942-50; Zagon I S, Am J. Physiol. 1996 271(3 Pt 2):R 780-6; Hytrek S D, Am J. Physiol. 1996 271(1 Pt 2):R 115-21; Zagon I S, Am J. Physiol. 1996; 271(3 Pt 2):R 511-8; Zagon I S, Brain Res. 1993 605(1):50-6; and Zagon I S, Cancer Lett. 1997112(2): 167-75, among other technical publications. These studies demonstrate, for example, that exogenously provided endorphins bind the zeta receptor and retard tumor growth. In addition to Met⁵-enkephalin, many other opioids and endorphins bind this receptor.

As used herein the term “sustained release formulation” and similar terms and phrases, relate generally to any formulation which, when mixed, blended, dissolved, comminuted, or incorporated with a pharmaceutically active agent for use in drug delivery, extends the effective duration of time over which the pharmaceutically active agent is bioavailable. The resulting product is synonymously termed a “composition”, a “pharmaceutical composition” or a “medicament” herein; these terms therefore relate to a sustained release composition, a sustained release pharmaceutical composition or a sustained release medicament as used herein. Although a sustained release formulation generally encompasses both enteric and parenteral administration, within the context of the present invention only parenteral uses are contemplated. In general, a composition, or a pharmaceutical composition, or a medicament, releases the pharmaceutically active agent over a period of time such that the length of time over which the active agent is bioavailable is greater than it would be in the absence of the formulation. Thus the bioavailability may be 1 day or longer, or about 2 days or longer, or about 4 days or longer, or about 1 week or longer, or about 10 days or longer, or about 2 weeks or longer, or about 20 days or longer, or about 3 weeks or longer, or about 4 weeks or longer, or about 5 weeks or longer, or about 6 weeks or longer, or about 7 weeks or longer, or about 8 weeks or longer, or about 9 weeks or longer, or about 10 weeks or longer, or about 11 weeks or longer, or about 12 weeks or longer, or about 13 weeks or longer, or even longer periods than any of the above. As used herein the term “controlled release” and similar terms and phrases are synonymous with “sustained release”.

The composition, pharmaceutical composition or medicament is generally a mixture, or a blend, or a solution, or a suspension, or a dispersion, or a comminution of one or more pharmaceutically active agents and a sustained release formulation which contains sufficient amounts of an active agent or agents that the concentration or amount of the agent or agents released is sufficient to exert a therapeutic effect throughout the period of sustained release. In the context of the present invention an active agent such as an enkephalin or a derivative thereof, an endorphin or a derivative thereof, a dynorphin or a derivative thereof, or a cancer chemotherapeutic agent, or any combination thereof, is released in sufficient quantity throughout substantially all of a predetermined period of sustained release that a sufficient proportion of cancer cells in the vicinity of the medicament binds the active agent to elicit an intended response throughout the period of sustained release.

A wide range of sustained release formulations is known, any of which may be employed in the present invention. By way of nonlimiting example, sustained release formulations may be gels, hydrogels, formulations applied by spray, bioadhesive formulations, and similar formulations. Additionally, sustained release formulations may be constituted of, or may include as one of several components, particles, beads, microparticles, nanoparticles, and similar formed entities that include a pharmaceutically active agent as a component. In general, a sustained release formulation used in the present invention is substantially biocompatible, or substantially biodegradable. As used herein “biocompatible” signifies that the components in the sustained release formulation are benign with respect to cells, internal tissues or internal organs of a subject. A benign component elicits no adverse reaction from the host subject, whether locally at a site of administration or application, or systemically as the component is resorbed by bodily fluids such as lymph, interstitial fluid, plasma, serum or blood. As used herein “biodegradable” signifies that a component is degraded in situ at the site of administration or application, or systemically, to provide degradation products that are either nontoxic metabolites in the cells of the subject, or nontoxic products that are readily eliminated from the subject, for example through the circulatory system and an elimination pathway. Biodegradation may occur spontaneously, such as by hydrolysis due to contact with an aqueous milieu within the subject, or it may be catalyzed by an enzymatic activity present in the aqueous milieu such as the interstitial fluid, lymph or serum.

As used herein, the term “conjugated” and similar terms and phrases relate to a composition, pharmaceutical composition or medicament in which a pharmaceutically active agent, or drug, or a chemical precursor to such a drug (termed a “prodrug” herein), is covalently linked to a moiety of a polymeric drug delivery substance. In many cases the bond which conjugates a drug to a polymeric substance is degraded in vivo at a relatively slow rate. Such slow degradation confers sustained release properties on the drug-polymer conjugate, or the prodrug-polymer conjugate, such that a conjugate becomes a sustained release pharmaceutical compositions. In other cases additional sustained release features may be incorporated into the conjugation chemistry, or into the polymeric substance, to provide a sustained release pharmaceutical composition of the invention.

Peptide Synthesis

An enkephalin, an endorphin, a dynorphin, or a derivative or fragment of any of them, may be synthesized and purified by methods widely known in the fields of peptide chemistry, protein chemistry, synthetic chemistry and other fields related to the field of the present invention. By way of nonlimiting example, any intended peptide may be produced by direct peptide synthesis using solid-phase techniques (see, e.g., Stewart et al., Solid-Phase Peptide Synthesis, W.H. Freeman Co., San Francisco, Calif. (1969); Bodanszky, M., et al., Principles of Peptide Synthesis, 2^{nd} Ed., Springer-Verlag, Inc., New York, N.Y. (1993)). In vitro peptide synthesis may be performed using manual techniques or by automation. Automated synthesis may be accomplished, for instance, using an Applied Biosystems Peptide Synthesizer (Foster City, Calif.) using the manufacturer's instructions.

In a nonlimiting example of a synthetic protocol, a peptide is synthesized on Pepsyn K polyamide-Kieselguhr resin using a Milligen 9050 PepSynthesizer (Milligen, Novato, Calif.) which is functionalized with ethylenediamine and onto which the acid-labile linker 4-(alpha-Fmoc-amino-2′,4′-dimethoxybenzyl) phenoxyacetic acid is coupled (Rink, Tetrahedron Lett. (1987) 28:3787). t-Butyl-based side-chain protection and Fmoc alpha-amino-protection is used. The guanidino-group of arginine is protected by the 2,2,5,7,8-pentamethylchroman-6-sulfonyl moiety. The imidazole group of histidine is protected by either t-Boc or trityl and the sulfhydryl group of cysteine is protected by a trityl group. Couplings are carried out using O-pentafluorophenyl esters except in the case of arginine where diisopropylcarbodiimide-mediated hydroxybenzotriazole ester formation is employed. Following cleavage of the completed peptide from the resin with trifluoroacetic acid in the presence of scavengers and extraction with diethylether, peptide products may be analyzed by C₁₈-reverse phase chromatography.

In an alternative example, a peptide may be synthesized on an Applied Biosystems (Foster City, Calif.) model 430A peptide synthesizer which is modified for accelerated Boc-chemistry solid phase peptide synthesis. See Schnoize, et al., Int. J. Peptide Protein Res., 90:180 (1992). 4-Methylbenz-hydrylamine (MBHA) resin (Peninsula, Belmont, Calif.) is used. The Boc amino acids (Bachem, Calif., Torrance, Calif.; Nova Biochem., La Jolla, Calif.) are used with the following side chain protection: Boc-Ala-OH, Boc-Arg(Tos)-OH, Boc-Asp(OcHex)-OH, Boc-Glu(OcHex)-OH, Boc-His(DNP)-OH, Boc-Val-OH, Boc-Leu-OH, Boc-Gly-OH, Boc-Gln-OH, Boc-Ile-OH, Boc-Lys(2ClZ)-OH, Boc-Thr(Bzl)-OH, and Boc-Ser(Bzl)-OH, where Boc designates t-butyloxycarbonyl, Tos designates tosyl, OcHex designates O-substituted cyclohexyl, DNP designates dinitrophenyl, 2ClZ designates 2-clorobenzyl, and Bzl designates benzyl. Boc groups are removed by treatment with 100% TFA. Boc amino acids are pre-activated with HBTU and DIEA in DMF and are coupled without prior neutralization of the peptide-resin TFA salt.

At the end of the assembly of the peptide chain, the resin is treated with a solution of 20% mercaptoethanol/10% DIEA in DMF to remove the DNP group on a His side chain, if present. The N-terminal Boc group is then removed by treatment with 100% TFA. The partially-deprotected peptide-resin was washed with DMF and DCM and dried under reduced pressure. The peptide is cleaved from the resin by stirring HF containing 1 mL of anisole and dithiothreitol at 0 deg C. The residue is washed with ether and extracted with 4N HOAc.

The peptide mixture in the aqueous extract is purified on a reversed-phase preparative high pressure liquid chromatography (HPLC) using a reversed phase Vydac™ C₁₈ column (Nest Group, Southborough, Mass.). Fractions containing pure product are combined and lyophilized to dryness. Identity is verified using mass spectrometry and amino acid analysis.

Sustained Release Formulations

Nonlimiting examples of sustained release formulations and sustained release compositions are exemplified in the selected patents and publications summarized herein.

U.S. Pat. No. 6,858,229 discloses a hydrogel precursor composition including (a) a polymer containing a water soluble polymer domain with at least two hydrophobic interacting groups attached thereto that forms a hydrogel under physiological conditions; and (b) a cyclodextrin protecting group that inhibits gel formation of said polymer. This patent further discloses that removal of the inhibitory substance permits the hydrogel to form. The hydrogel and its precursor may additionally contain a drug. The hydrogels described in this patent may be formed in contact with a tissue.

U.S. Pat. No. 6,673,767 discloses compositions that include a biocompatible polymer, a biocompatible solvent having low water miscibility that forms a viscous gel with the polymer and limits water uptake by an implant formed from the polymer, and a beneficial agent. This patent states that polymers may be polylactides or other polyesters, polyanhydrides, polyamines, polyurethanes, polyesteramides, polyorthoesters, polydioxanones, polyacetals, polyketals, polycarbonates, polyorthocarbonates, polyphosphazenes, succinates, poly(malic acid), poly(amino acids), polyvinylpyrrolidone, polyethylene glycol, polyhydroxycellulose, chitin, or chitosan. With suitable nonaqueous solvents these compositions are injectable gels.

U.S. Pat. No. 6,689,390 discloses a lipid or other hydrophobic or amphiphilic compound integrated into a polymeric matrix for drug delivery to alter drug release kinetics. The patent states that diffusion of water into the microparticle and diffusion of solubilized drug out of the matrix is modified. The integrated hydrophobic compound also prolongs degradation of hydrolytically unstable polymers forming the matrix, further delaying release of encapsulated drug. The matrix includes preferred biocompatible, hydrolytically unstable polymers such as polyhydroxy acids including polylactic acid-co-glycolic acid, polylactide, polyglycolide or polylactide co-glycolide, which may be conjugated to polyethylene glycol.

U.S. Pat. No. 6,692,759 discloses an implantable sustained release system using either biodegradable polymers or nonbiodegradable polymers.

U.S. Pat. No. 6,733,787 discloses a matrix including a biocompatible, bioerodable polymer, wherein the polymer includes a thermoplastic lactide-containing terpolymer of monomer units derived from lactic acid, glycolic acid, and either caprolactone or valerolactone, The matrix or adhesive can further include a filler or a bioactive agent, or both.

U.S. Pat. No. 6,805,876 discloses phosphate based biodegradable polymers containing repeat units derived from cyclic phosphate monomers and optionally further containing repeat units derived from lactide or caprolactone monomers. The resulting polymers may be fashioned into articles or microspheres. The patent states that the articles or microspheres may be used for the controlled release of a biologically active substance.

U.S. Pat. No. 6,773,714 discloses a flowable composition suitable for use as a controlled release implant; the patent discloses a biodegradable thermoplastic homopolymer or copolymer of lactide, glycolide, or caprolactone; and a biocompatible polar aprotic solvent that is miscible to dispersible in aqueous medium or body fluid; and leuprolide acetate in the particular invention disclosed.

U.S. Pat. No. 6,828,412 discloses a biodegradable polymer containing an acid labile, pH dependent backbone incorporating a cis-aconityl group therein. This group is designed to remain stable in plasma at neutral pH, but to degrade intracellularly by hydrolysis in the more acidic environment of an endosome or lysosome. According to this patent, a therapeutic agent such as a cancer chemotherapeutic drug may be delivered by conjugation to the polymer, such that it is released by a degradative process including the process indicated above.

U.S. Pat. No. 6,803,438 discloses a polymer having a polydispersity of less than 1.2; the polymer is preferably an activated polyacrylate ester that is prepared by Controlled Radical Polymerization. According to the patent, these polymers are designed to be derivitisable and may be used to form polymer-drug conjugates having improved biological profile.

U.S. Pat. No. 6,624,245 discloses a biocompatible gel which may be prepared in situ, such as at a selected site within a patient's body. The gel is formed by admixing biocompatible crosslinking components A having sulfhydryl groups and a biocompatible crosslinking component B having sulfhydryl-reactive groups such that the sulfhydryl-reactive groups are capable of covalent reaction to form a gel in less than one minute. Exemplary uses include tissue augmentation, biologically active agent delivery, or bioadhesion. The polymer may be synthetic such as polyalkylene oxides, polyols, polyacrylates, poly(N-vinyl lactam)s; semi-synthetic, or naturally occurring, such as collagen.

U.S. Pat. No. 5,936,035 discloses a bioadhesive composition containing fibrillar collagen, a fiber disassembly agent, and a multifunctionally activated synthetic hydrophilic polymer, wherein the collagen and synthetic polymer covalently bind to form a collagen-synthetic polymer conjugate. The collagen-based bioadhesive compositions may also be formulated to contain biologically active agents such as growth factors in order to facilitate adhesion of tissues or healing of adhered tissues.

U.S. Pat. No. 5,702,717 discloses an injectable biodegradable block copolymeric drug delivery liquid having reverse thermal gelation properties containing an aqueous solution that has uniformly contained within it (a) an effective amount of a drug intimately contained in (b) a biodegradable block copolymer that includes a hydrophobic A polymer block made from poly(alpha-hydroxy acids) or poly(ethylene carbonates), and a hydrophilic B polymer block including a polyethylene glycol. The patent states that the solution remains liquid at a temperature below the lower critical solution temperature of the block copolymer.

U.S. Pat. No. 6,201,072 discloses an aqueous biodegradable polymeric drug delivery composition possessing reverse thermal gelation properties that includes an aqueous phase containing (a) an effective amount of a drug; and (b) a biodegradable ABA- or BAB-type tri-block polymer. According to this patent the ABA triblock includes a hydrophobic A polymer block containing a biodegradable polyester, and a hydrophilic B polymer block containing a polyethylene glycol (PEG).

U.S. Pat. Nos. 6,287,588 and 6,589,549 disclose a dual phase polymeric agent-delivery composition including: (a) a continuous biocompatible gel phase, (b) a discontinuous particulate phase comprising defined microparticles; and (c) an agent to be delivered contained in both the continuous biocompatible gel phase and the discontinuous particulate phase.

U.S. Pat. No. 6,309,633 discloses a drug-oligomer conjugate including a therapeutic drug moiety; hydrophilic moieties independently selected from among straight or branched PEG polymers, and sugars; lipophilic moieties, independently selected from among alkyl groups having 2-24 carbon atoms, cholesterol, adamantane and fatty acids. According to this patent the therapeutic drug moiety has at least one available moiety for conjugation selected from among —NH2; —OH and —SH, and at least one of the available moieties is conjugated to the hydrophilic—lipophilic (H-L) moiety; and the H-L bond(s) are hydrolyzable and the drug-lipophilic bond(s), when present, are hydrolysable.

U.S. Patent Application Publication 20040156819 discloses block copolymers of polyesters and polyalkylene glycols used to prevent postsurgical tissue adhesion. The copolymers form gels in aqueous solution. A secondary use of the block copolymers is to deliver bioactive compositions to a site of activity within a patient's body.

U.S. Patent Application Publication 20010009662 discloses polymeric materials in the form of block copolymers such as AB. The polymers may be manufactured in film form and other solid structures such as rods, cylinders, and to form foams, dispersions, viscous solutions, liquid polymers, pastes, sprays or gels adaptable for use in a wide range of applications. They may be effective for delivering bioactive agents. According to this publication the A block contains alpha-hydroxyacid units or a related acid, ester or similar compound, and, in certain embodiments the B block is preferably a hydroxyl, carboxylic acid or amine terminated poly(oxyalkylene) block.

U.S. Pat. No. 6,870,012 discloses a polymeric composition exhibiting reverse thermal gelation properties. The polymeric composition has the general structure:

{[Aₙ (BCB)Aₙ]E}ₘ, where A is a polyester unit, B is a poly(ethylene oxide) unit, C is a poly(propylene oxide) unit, and E is a chain extender unit. The viscosity at a final higher temperature is more than twice the viscosity of the composition at an initial temperature at least 10° C. lower than the final temperature. The polymeric composition may be combined with a bioactive agent.

U.S. Pat. No. 5,591,709 discloses a wound-healing gel formulation containing a growth-promoting hormone and a naturally occurring or synthetic polymer exemplified by agarose, gelatin, collagen, various synthetic hydrophilic vinyl polymers or a hydrophilic cellulose polymer.

U.S. Pat. No. 6,833,408 discloses a method for tissue repair utilizing a readily crosslinkable, biocompatible, adhesive composition to secure a surgically acceptable patch to the damaged tissue. The adhesive composition is comprised of a hydrophilic polymer, a crosslinkable component A having m nucleophilic groups, and a crosslinkable component B having n electrophilic groups capable of reaction with the m nucleophilic groups to form covalent bonds. Admixture of components A and B in an aqueous medium results in crosslinking of the composition to give a biocompatible, nonimmunogenic, crosslinked matrix. In many embodiments the hydrophilic polymer is a form of collagen. According to this patent the composition may provide controlled release of biologically active agents.

U.S. Pat. No. 6,624,245 discloses a method for the rapid formation of a biocompatible gel, and may be carried out in situ, i.e., within a patient's body. The method involves admixing a biocompatible crosslinking component A having m sulfhydryl groups and a biocompatible crosslinking component B having n sulfhydryl-reactive groups. According to this patent, under crosslinking conditions the components form a gel in less than one minute. Exemplary uses include tissue augmentation, biologically active agent delivery, and bioadhesion.

U.S. Pat. No. 6,534,591 discloses crosslinked polymer compositions including a first synthetic polymer containing multiple nucleophilic groups covalently bound to a second synthetic polymer containing multiple electrophilic groups. The compositions may further comprise other components, such as naturally occurring polysaccharides or proteins (such as glycosaminoglycans or collagen) and/or biologically active agents. Among several uses, the patent states that the compositions are useful as bioadhesives.

Other drug delivery systems for sustained release are injected as small particles, microspheres, microcapsules, or nanoparticles. For example, U.S. Pat. No. 5,019,400 describes the preparation of controlled release biodegradable microspheres made of various polyesters, polycarbonates, polyamides, polyanhydrides, polyamino acids, polyortho esters, polyacetals, polycyanoacrylates, or polyurethanes, via a very low temperature casting process.

U.S. Pat. No. 5,143,661 discloses compositions including a microencapsulated core material wherein the microcapsules are prepared by phase separation microencapsulation employing a volatile silicone fluid as a hardening agent. Among the encapsulating polymers which can be utilized, this patent identifies polyglycolide, polylactide (L or DL), poly (glycolide-co-l-lactide), poly (glycolide-co-dl-lactide), other polyhydroxyl esters, poly(ortho esters), poly(anhydrides), poly(amide esters), poly(alkylene tartrate), poly(alkylene fumarate), cellulose based polyurethanes, ethyl cellulose, methyl cellulose, hydroxypropyl cellulose, and other cellulose derivatives.

U.S. Pat. No. 6,117,455 discloses a sustained-release microcapsule which is obtained by selecting a dispersion of an amorphous water-soluble pharmaceutical agent having a particle size of from 1 nm-10 μm in a solution of a polymer in an organic solvent; dispersing the dispersion of amorphous water-soluble pharmaceutical agent in an aqueous phase to prepare a solid-in-oil-in water (s/o/w) emulsion; and subjecting the s/o/w emulsion to in-water drying.

U.S. Pat. No. 5,417,982 discloses a controlled release microsphere formulation for use with drugs. The drug is suspended in a polymer matrix that is formed from at least two highly water soluble biodegradable polymers. The microspheres are coated with a (d, 1-lactide-glycolide) copolymer.

U.S. Pat. No. 5,718,921 discloses microspheres including a polymer and a drug dispersed within each microsphere, whereby the microspheres are formed in the absence of water, and the polymer is selected from the group of biodegradable polymers consisting of polyanhydrides, polyorthoesters, polylactic acid polymers, and combinations thereof, preferably polyanhydrides, and the drug is a biological or a labile drug. According to methods of this patent alone or in combination with other methods multi-walled microspheres having each wall degrading at a different rate or containing different drugs can be manufactured.

U.S. Pat. No. 5,731,005 discloses a sustained release delivery composition for ocular use containing hydrogel microspheres containing the pharmaceutically active agent, in an aqueous carrier. According to this patent the hydrogel microspheres include cross-linked macromolecules and macromolecules that possess an affinity for the agent.

U.S. Pat. No. 6,306,406 discloses a drug delivery system for controlled release of a protein or polypeptide including a hydrophobic biodegradable polymer and a protein or polypeptide. The drug delivery system may be prepared by a polymer precipitation technique or a microsphere technique. The polymer may be a biodegradable hydrophobic polymer selected from among polyglycolic acid, polylactic acid, copolymers of glycolic acid and L- or D,L-lactic acid, and copolymers of glycolide and L- or D,L-lactide.

U.S. Pat. No. 6,511,477 discloses therapeutic compounds containing very small capsules which can be injected into body tissue, particularly the heart. The capsules include an encapsulating layer which surrounds a therapeutic agent. According to this patent, after injection, the encapsulating layer degrades or dissolves, and the therapeutic agent is released within the heart. The therapeutic agent may be one of any number of known agents such as anti-arrhythmic drugs, gene therapy solutions, and macromolecules intended to have either acute or long-term effects on the heart.

U.S. Pat. No. 6,623,762 discloses a pharmaceutical composition wherein a drug is in soluble glass composite microspheres of sugar and calcium phosphate. The microspheres are suspended in a biocompatible anhydrous liquid whereby the drug is protected against dissolution while remaining surrounded by anhydrous liquid thereby extending the duration of action of the drug long after injection by slowly releasing the drug or vaccine into the patient's circulatory system.

U.S. Pat. No. 6,517,859 discloses polymeric microspheres as injectable drug-delivery vehicles for use to deliver bioactive agents to sites within the central nervous system, and for the stimulation of nerve fiber growth by implanting such microspheres within the central nervous system of a patient. According to this patent the microspheres include a polymer chosen from among poly(lactide-co-caprolactone) copolymer, polycaprolactone, and polyhydroxybutyrate-polyhydroxyvalerate copolymer, said polymer (1) being permeable to the neuroactive agent, (2) being biocompatible with the tissues of the central nervous system, and (3) having kinetic characteristics that may be manipulated to allow for the permeation of the neuroactive agent through the polymer at a controlled rate and a predetermined period of time.

As used herein “combination” and similar terms and phrases relate generally to a structure that may be described as a physical juxtaposition of two or more entities: a) a component of the body of a subject and b) a medicament of the invention, and possibly c) with additional entities. Physical juxtaposition relates in nonlimiting ways to actual physical contact of the components, or to mutual positioning in which the components are as close as possible to each other, given the interposition of an intervening biological structure such as a membrane, a tendon, a cartilage or a similar anatomical structure situated between a component and a medicament. The component of the body may be as small as a cell, or it may be a tissue comprised of cells. A characteristic of the physical juxtaposition intended in this invention is that an active agent released from the medicament have a minimal diffusion path to reach a cell containing a target of an enkephalin, and endorphin, a dynorphin, or a derivative of any of them, such as a zeta receptor, or an analogous target of a cancer chemotherapeutic agent (target cell). In a combination of the invention, therefore, a substantial proportion of the active agent comprised within the medicament ultimately reaches a target cell, binds the zeta receptor or other target structure comprised therein, and elicits the characteristic growth inhibitory response.

Sustained Release Compositions

A composition, pharmaceutical composition, or medicament of the invention includes a) one or both of a peptide ligand that binds a zeta receptor and at least one cancer chemotherapeutic agent; and b) a sustained release formulation. In certain embodiments of the invention the medicament includes a peptide ligand of a zeta receptor, which may be an enkephalin, a fragment of an enkephalin, or a derivative thereof. In additional embodiments the medicament includes a zeta receptor ligand that may be an endorphin, a fragment of an endorphin, or a derivative thereof. In still further embodiments the medicament includes a zeta receptor ligand that may be a dynorphin, a fragment of a dynorphin, or a derivative thereof. In yet additional embodiments the medicament includes one or more cancer chemotherapeutic agents, such as, by way of nonlimiting example, the cancer chemotherapeutic agents disclosed herein (see below).

In still additional embodiments the medicament includes both a peptide ligand of a zeta receptor and at least one cancer chemotherapeutic agent. The peptide component may be an enkephalin, a fragment of an enkephalin, or a derivative thereof; or an endorphin, a fragment of an endorphin, or a derivative thereof; or a dynorphin, a fragment of a dynorphin, or a derivative thereof.

The one or more cancer chemotherapeutic agents may be chosen, by way of nonlimiting example, from among asparaginase, hydroxyurea, altretamine, bleomycin, dactinomycin, doxorubicin, etoposide, teniposide, plicamydin, nitrogen mustards such as chlorambucil, cyclophosphamide, isofamide, mechlorethamine, melphalan, uracil mustard; an aziridine such as thiotepa, methanesulphonate esters such as busulfan, nitroso ureas such as carmustine, lomustine, streptozocin; platinum complexes, such as cisplatin and carboplatin; mitomycin, procarbazine, dacarbazine; amsacrine, daunorubicin, adriamycin, idarubicin, mitoxantrone; etoposide; teniposide, folate antagonists such as methotrexate and trimetrexate; pyrimidine antagonists, such as fluorouracil, fluorodeoxyuridine, CB3717, azacytidine, cytarabine, and floxuridine; purine antagonists such as mercaptopurine, 6-thioguanine, fludarabine, pentostatin, vincristine, vinblastine, paclitaxel, other taxanes, estrogens, conjugated estrogens, ethinyl estradiol and diethylstilbesterol, chlortrianisen and idenestrol; progestins such as hydroxyprogesterone caproate, medroxyprogesterone, and megestrol; androgens such as testosterone, testosterone propionate; fluoxymesterone, and methyltestosterone, prednisone, dexamethasone, methylprednisolone, prednisolone, leuprolide acetate, goserelin acetate, tamoxifen, flutamide; mitotane; and aminoglutethimide. Therapeutic uses for many of these agents are shown in Table 3.

TABLE 3
Neoplastic Diseases¹ for which Exemplary Chemotherapeutic agents are
Indicated

| Class of Agent | Diseases treated by Agent² | Type of Agent | Name of Agent |
|---|---|---|---|
| Alkylating Agents | Hodgkin's disease, non-Hodgkin's lymphomas | Nitrogen Mustards | Mechlorethamine |
| | Acute and chronic lymphocytic leukemias, Hodgkin's | | (HN₂) |
| | disease, non-Hodgkin's lymphomas, multiple myeloma, | | Cyclophosphamide |
| | neuroblastoma, breast, ovary, lung, Wilms' tumor, cervix, | | Ifosfamide |
| | testis, soft-tissue sarcomas | | |
| | Multiple myeloma, breast, ovary | | Melphalan |
| | Chronic lymphocytic leukemia, primary | | Chlorambucil |
| | macroglobulinemia, Hodgkin's disease, non-Hodgkin's | | |
| | lymphomas | | |
| | Prostate | | Estramustine |
| | Ovary | Ethylenimines and | Hexamethylmelamine |
| | | Methylmelamines | |
| | Bladder, breast, ovary | | Thiotepa |
| | Chronic granulocytic leukemia | Alkyl Sulfonates | Busulfan |
| | Hodgkin's disease, non-Hodgkin's lymphomas, primary | Nitrosoureas | Carmustine |
| | brain tumors, multiple myeloma, malignant melanoma | | |
| | Hodgkin's disease, non-Hodgkin's lymphomas, primary | | Lomustine |
| | brain tumors, small-cell lung | | |
| | Primary brain tumors, stomach, colon | | Semustine |
| | Malignant pancreatic insulinoma, malignant carcinoid | | Streptozocin |
| | Malignant melanoma, Hodgkin's disease, soft-tissue sarcomas | Triazenes | Dacarbazine |
| | | | Procarbazine |
| | | | Aziridine |
| Antimetabolites | Acute lymphocytic leukemia, choriocarcinoma, mycosis | Folic Acid Analogs | Methotrexate |
| | fungoides, breast, head and neck, lung, osteogenic sarcoma | | Trimetrexate |
| | Breast, colon, stomach, pancreas, ovary, head and neck, | Pyrimidine Analogs | Fluorouracil |
| | urinary bladder, premalignant skin lesions (topical) | | Floxuridine |
| | Acute granulocytic and acute lymphocytic leukemias | | Cytarabine |
| | | | Azacitidine |
| | Acute lymphocytic, acute granulocytic, and chronic | Purine Analogs and | Mercaptopurine |
| | granulocytic leukemias | Related Inhibitors | |
| | Acute granulocytic, acute lymphocytic, and chronic | | Thioguanine |
| | granulocytic leukemias | | |
| | Hairy cell leukemia, mycosis fungoides, chronic | | Pentostatin |
| | lymphocytic leukemia | | |
| | Chronic lymphocytic leukemia, Hodgkin's and | | Fludarabine |
| | non-Hodgkin's lymphomas, mycosis fungoides | | |
| Natural Products | Hodgkin's disease, non-Hodgkin's lymphomas, breast, testis | Vinca Alkaloids | Vinblastine (VLB) |
| | Acute lymphocytic leukemia, neuroblastoma, Wilms' | | Vincristine |
| | tumor, rhabdomyosarcoma, Hodgkin's disease, | | |
| | non-Hodgkin's lymphomas, small-cell lung | | |
| | Vinca-resistant acute lymphocytic leukemia, chronic | | Vindesine |
| | myelocytic leukemia, melanoma, lymphomas, breast | | |
| | Testis, small-cell lung and other lung, breast, Hodgkin's | Epipodophyllotoxins | Etoposide |
| | disease, non-Hodgkin's lymphomas, acute granulocytic | | Teniposide |
| | leukemia, Kaposi's sarcoma | | |
| | Choriocarcinoma, Wilms' tumor, rhabdomyosarcoma, | Antibiotics | Dactinomycin |
| | testis, Kaposi's sarcoma | | |
| | Acute granulocytic and acute lymphocytic leukemias | | Daunorubicin |
| | Soft-tissue, osteogenic, and other sarcomas; Hodgkin's | | Doxorubicin |
| | disease, non-Hodgkin's lymphomas, acute leukemias, | | 4′-Deoxydoxorubicin |
| | breast, genitourinary, thyroid, lung, stomach, | | |
| | neuroblastoma | | |
| | Testis, head and neck, skin, esophagus, lung, and | | Bleomycin |
| | genitourinary tract; Hodgkin's disease, non-Hodgkin's | | |
| | lymphomas | | |
| | Testis, malignant hypercalcemia | | Plicamycin |
| | | | Mitomycin |
| | Stomach, cervix, colon, breast, pancreas, bladder, head and | | |
| | neck | | |
| | Acute lymphocytic leukemia | Enzymes | L-Asparaginase |
| | | Taxanes | Docetaxel |
| | Breast, ovarian | Taxoids | Paclitaxel |
| | Hairy cell leukemia, Kaposi's sarcoma, melanoma, | Biological Response | Interferon Alfa |
| | carcinoid, renal cell, ovary, bladder, non-Hodgkin's | Modifiers | |
| | lymphomas, mycosis fungoides, multiple myeloma, chronic | | |
| | granulocytic leukemia | | |
| | Investigational | | Tumor Necrosis |
| | | | Factor |
| | Investigational | | Tumor-Infiltrating |
| | | | Lymphocytes |
| Miscellaneous Agents | Testis, ovary, bladder, head and neck, lung, thyroid, cervix, | Platinum Coordination | Cisplatin |
| | endometrium, neuroblastoma, osteogenic sarcoma | Complexes | Carboplatin |
| | Acute granulocytic leukemia, breast | Anthracenedione | Mitoxantrone |
| | Chronic granulocytic leukemia, polycythemia vera, | Substituted Urea | Hydroxyurea |
| | essential thrombocytosis, malignant melanoma | | |
| | Hodgkin's disease | Methyl Hydrazine | Procarbazine |
| | Derivative | | |
| | Adrenal cortex | Adrenocortical Suppressant Mitotane | |
| | Breast | | Aminoglutethimide |
| Hormones and | Acute and chronic lymphocytic leukemias, non-Hodgkin's | | |
| Antagonists | lymphomas, Hodgkin's disease, breast | | |
| Adrenocorti-costeroids | Endometrium, breast | Progestins | Hydroxy-progesterone |
| Prednisone | | | caproate |
| | | | Medroxy-progesterone |
| | | | acetate |
| | | | Megestrol acetate |
| | Breast, prostate | Estrogens | Diethylstil-bestrol |
| | | | Ethinyl estradiol |
| | Breast | Antiestrogen | Tamoxifen |
| | Breast | Androgens | Testosterone |
| | | | propionate |
| | | | Fluoxymesterone |
| | Prostate | Antiandrogen | Flutamide |
| | Prostate, Estrogen-receptor-positive breast | Gonadotropin-releasing | Leuprolide |
| | | hormone analog | Goserelin |

¹Adapted from Calabresi, P., and B. A. Chabner, “Chemotherapy of Neoplastic Diseases” Section XII, pp 1202-1263 in: Goodman and Gilman's The Pharmacological Basis of Therapeutics, Eighth ed., 1990 Pergamon Press, Inc.; and Barrows, L. R., “Antineoplastic and Immunoactive Drugs”, Chapter 75, pp 1236-1262, in: Remington: The Science and Practice of Pharmacy, Mack Publishing Co. Easton, PA, 1995.; both references are incorporated by reference herein.
# in particular for treatment protocols.
²Neoplasms are carcinomas unless otherwise indicated.

Nonlimiting examples of monoclonal antibody cancer therapeutic agents that may be used in the medicaments of the invention include the following:
Rituximab (Rituxan): Rituximab is used to treat B cell non-Hodgkin's lymphoma. It contains a monoclonal antibody against the CD20 antigen, found on B cells.
Trastuzumab (Herceptin): Trastuzumab is an antibody against the HER2 protein, which is present in large numbers on cells in some cases of breast cancer. It is used to treat advanced cases of the disease.
Alemtuzumab (Campath): Alemtuzumab is an antibody against the CD52 antigen, which is present on both B cells and T cells. It is used to treat B cell chronic lymphocytic leukemia (B-CLL) in patients who have already had chemotherapy.
Cetuximab (Erbitux): Cetuximab is an antibody against the EGFR protein, which is present in high amounts on some tumor cells. It is used along with the chemotherapy drug irinotecan to treat advanced colorectal cancer.
Bevacizumab (Avastin): Bevacizumab works against the VEGF protein, which normally helps tumors develop new blood vessels in order to get nutrients. This antiangiogenesis therapy is used along with chemotherapy to treat metastatic colorectal cancer.
Ibritumomab (Zevalin): a Radioimmunotherapeutic agent conjugated with Y-90, approved for use against relapsed or refractory low-grade follicular or transformed B-cell non-Hodgkins lymphoma.
Tositumomab (Bexxar) a Mab-I-131 radionuclide conjugate directed against non-Hodgkin's lymphoma b.

The sustained release formulation included in the medicament may be any of a wide range of formulations. In general, sustained release formulations employed in the compositions, combinations and methods of the present invention are biocompatible, and in many embodiments are biodegradable. Nonlimiting examples of such formulations include gels, hydrogels, formulations applied by spray, bioadhesive formulations, and similar formulations, certain examples of which have been described hereinabove. Thus, by way of nonlimiting example, a sustained release formulation may include a natural polymer, such as a protein, a polysaccharide, a mucopolysaccharide, and the like. A protein component may be an albumin, a fibrinogen or a fibrin or solubilized derivative thereof, any of the several types of collagen in both fibril form or disaggregated, any of the several types of collagen that are atelocollagens, any of the several types of collagen that have been partially degraded in molecular weight, various gelatins derived from the several types of collagen, and the like. Polysaccharides include cellulose, cellulose derivatives such as alkyl celluloses, carboxyalkyl celluloses, glycogen, biocompatible vegetable gums, chitin, chitosan, biocompatible seaweed gums, chondroitin sulfate, hyaluronic acid, and the like. Nonlimiting examples of sustained release formulations that may be used in the compositions of the present invention include those disclosed in patents and patent application publications cited herein, and incorporated by reference herein in their entireties.

Further, by way of nonlimiting example, a sustained release formulation may include a synthetic polymer, such as a homopolymers or a copolymer, wherein the copolymer may be a homogeneously copolymerized copolymer or a block copolymer. Components of synthetic polymers may be chosen, by way of nonlimiting example, from among poly(hydroxyacyl)esters; poly(lactone)esters; various polyvinyl derivatives including polyvinylpyrrolidone, polyvinyl alcohol, and the like; polyethyleneoxide and homologous polyalkyleneoxides, both capped and uncapped; polycarbonates; polyanhydrides; polyamines; polyurethanes; polyesteramides; polyorthoesters; polydioxanones; polyacetals, polyketals; polycarbonates; polyorthocarbonates; polyphosphazenes; succinates; poly(malic acid); and poly(amino acids).

A sustained release formulation used in a medicament of the invention may be comprised of a single polymer, or a combination or mixture of more than one polymer.

A polymer or copolymer chosen for use in a medicament of the invention is to be biocompatible. In many embodiments the polymer additionally is biodegradable. A polymer used in a medicament may be formulated in an aqueous medium, or a dispersing solvent may include biocompatible nonaqueous solvents or cosolvents according to the properties of the chosen polymer, to permit the composition to be employed as a gel, a hydrogel, a formulation applied by spray, a bioadhesive formulation, or a similar formulation.

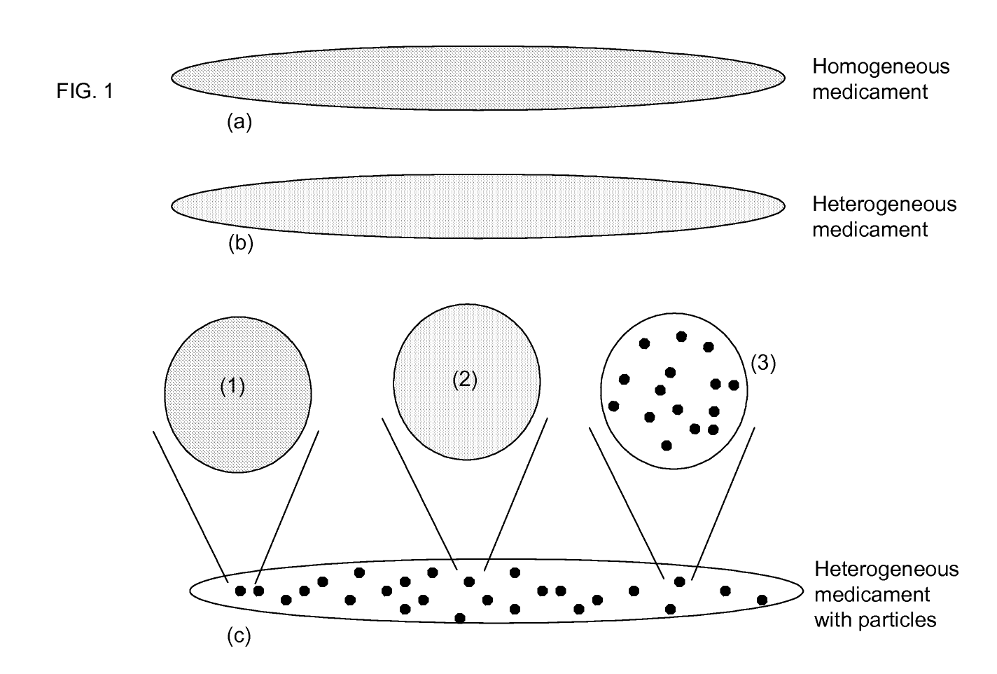

The one or more pharmaceutical active agents included in the pharmaceutical composition may be incorporated as a solution, a dispersion, a suspension, a comminution, or any similar composition, according to the respective properties of the active agent and the sustained release formulation. A schematic representation of an embodiment of a pharmaceutical composition in which an active agent is incorporated as a solution is shown in FIG. 1, panel (a); and a schematic representation of an embodiment in which an active agent is incorporated as a dispersion, a suspension, or a comminution is shown in FIG. 1, panel (b).

Additionally, a sustained release composition or medicament may be constituted of, or may include as one of several components, particles, beads, microparticles, nanoparticles, and similar formed entities that include a pharmaceutically active agent as a component. A schematic representation of embodiments of a sustained release composition including a particle, microparticle or nanoparticle is shown in FIG. 1, panel (c). In panel (c), a particle in which an active agent is incorporated as a solution is shown in the blowup of a particle in (1). A particle in which an active agent is included as a dispersion, a suspension, or a comminution of relatively small insoluble solids is shown in the blowup in (2), and an example in which an active agent is included in a particle as a relatively large insoluble solid is shown in the blowup in (3). The representations in FIG. 1 are nonlimiting; alternative or equivalent designs of pharmaceutical compositions are included within the scope of the present invention.

Furthermore, a sustained release composition or medicament may contain any of the pharmaceutically active agents disclosed herein, or a precursor thereof that gives rise to the pharmaceutically active agent in situ, conjugated to a polymer component of a sustained release formulation.

In general, a composition, or a pharmaceutical composition, or a medicament, releases the pharmaceutically active agent over a period of time such that the length of time over which the active agent is bioavailable is greater than it would be in the absence of the formulation. Thus the bioavailability may endure 1 day or longer, or about 2 days or longer, or about 4 days or longer, or about 1 week or longer, or about 10 days or longer, or about 2 weeks or longer, or about 20 days or longer, or about 3 weeks or longer, or about 4 weeks or longer, or about 5 weeks or longer, or about 6 weeks or longer, or about 7 weeks or longer, or about 8 weeks or longer, or about 9 weeks or longer, or about 10 weeks or longer, or about 11 weeks or longer, or about 12 weeks or longer, or about 13 weeks or longer, or even longer periods than any of the above.

A medicament of the invention may furthermore be composed of more than one composition, each of which includes one or both of a peptide ligand that binds a zeta receptor and at least one cancer chemotherapeutic agent, and a sustained release formulation that differs from the others. By way of nonlimiting example, a first pharmaceutical composition including one or both of a first peptide ligand that binds a zeta receptor and at least one first cancer chemotherapeutic agent, and a first sustained release formulation, may be combined with a second pharmaceutical composition including one or both of a second peptide ligand that binds a zeta receptor and at least one second cancer chemotherapeutic agent, and a second sustained release formulation. The various component pharmaceutical compositions may include different active agents, and may be characterized by having different sustained release formulations whose release kinetics may be the same, or may that may be similar, or that may differ substantially from each other. Thus, by way of nonlimiting example, a gel composition including a peptide ligand of a zeta receptor and having relatively rapid release kinetics, may be combined or admixed with a microsphere composition including one or more cancer chemotherapeutic agents and having relatively slow release kinetics. Alternatively, as another example, a gel composition including a first set of cancer chemotherapeutic agents and having relatively rapid release kinetics may be combined with a microcapsule including a second set of cancer chemotherapeutic agents having relatively slow release kinetics. Use of combined medicaments as described above permits addressing different molecular targets within a cancer cell or a precancerous cell, thereby increasing the likelihood of inducing death of the cancer cell and/or the precancerous cell, and optimizing the likelihood of remission of the cancer. Any of the component compositions in the combined medicament described above may release its pharmaceutical active agent in 1 day or greater, or about 2 days or greater, or about 4 days or greater, or about 1 week or greater, or about 10 days or greater, or about 2 weeks or greater, or about 20 days or greater, or about 3 weeks or greater, or about 4 weeks or greater, or about 5 weeks or greater, or about 6 weeks or greater, or about 7 weeks or greater, or about 8 weeks or greater, or about 9 weeks or greater, or about 10 weeks or greater, or about 11 weeks or greater, or about 12 weeks or greater, or about 13 weeks or greater, or even longer periods than any of the above.

Preparation of a Pharmaceutical Composition

A composition, pharmaceutical composition, or medicament of the invention may be prepared by incorporating a pharmaceutically active agent (also termed a beneficial agent, a substance, or a bioactive agent below), such as a peptide ligand of a zeta receptor, or a cancer chemotherapeutic agent, or both, into a mixture, blend, or solution of components that are included in a sustained release formulation, according to detailed procedures widely known in fields such as pharmaceutical chemistry, drug delivery technology, and other fields related to the field of the present invention.

By way of nonlimiting example, U.S. Pat. No. 6,673,767 discloses preparing an injectable gel composition by mixing a biocompatible polymer and suitable solvent to form a viscous gel; dispersing or dissolving a beneficial agent in an emulsifying agent; and mixing the result with the viscous gel resulting in a dispersed droplet phase of the emulsifying agent with the beneficial agent in the viscous gel. Alternatively, U.S. Pat. No. 6,689,390 discloses producing microparticles and related matrices by spray drying, melt extrusion, compression molding, fluid bed drying, solvent extraction, hot melt encapsulation, or solvent evaporation. A hydrophobic compound must be dissolved or melted with a disclosed polymer or dispersed as a solid or a liquid in a solution of the polymer, prior to forming the matrix. As a result, the hydrophobic (or amphiphilic) compound is mixed throughout the matrix. The active agent can be incorporated into the matrix as solid particles, as a liquid or liquid droplets, or by dissolving the agent in the polymer solvent.

Still further, U.S. Pat. No. 6,692,759 discloses a method for preparing an implantable device for a sustained delivery of a substance within a body of a human or an animal subject, including dissolving a biocompatible polymer in a suitable solvent solution to produce a polymer-solvent solution; adding the substance to the polymer-solvent solution to produce a polymer-solvent solution-substance admixture; drying the polymer-solvent solution-substance admixture to form a substantially dry mass; and further manipulations to achieve a desired consistency and/or shape. Yet additionally U.S. Pat. No. 6,733,787 discloses preparation of matrices containing preferred polyester ionomers such as those made up of lactide, glycolide and caprolactone or valerolactone, or alternatively including certain poly(amino acids) that exhibit adhesive properties toward connective tissue. In the matrix, the polymer may be combined with a bioactive agent, one or more fillers, or both, prepared by using standard formulation techniques such as mixing and compressing and microencapsulation. Furthermore, U.S. Pat. No. 6,805,876 discloses formation of biodegradable medical implant devices and drug delivery products melt processing of the patented polymer described in this patent using conventional extrusion or injection molding techniques, or by dissolving in an appropriate solvent, followed by formation of the device, and subsequent removal of the solvent by evaporation or extraction.

Additionally, U.S. Pat. No. 6,773,714 discloses a method for forming a flowable composition for use as a controlled release implant, comprising the step of mixing, in any order:

(a) a biodegradable thermoplastic homopolymer or copolymer of lactide, glycolide, or caprolactone, that includes a monofunctional alcohol or a diol residue; (b) a biocompatible polar aprotic solvent that is miscible to dispersible in aqueous medium or body fluid; and (c) leuprolide acetate. Yet further U.S. Pat. No. 6,828,412 discloses a method for releasing a bioactive agent comprising the steps of a) introducing a conjugate that has been synthesized by attaching a drug directly or through a linker to the backbone of a particular polymer disclosed in this patent to an environment having a pH of less than 6.5 (such as may occur in a lysosomes), and b) cleaving the bioactive agent from the linker group by acid or enzymatic hydrolysis. Additionally, U.S. Pat. No. 6,803,438 discloses methods for conjugating an amine-containing compound to, for example, a homopolymeric drug conjugate precursor, poly(methacryloxy succinimide).

Still further U.S. Pat. No. 6,309,633 discloses that an oligomer can be coupled to a peptide drug by converting the free hydroxyl moiety of the oligomer to the N-hydroxysuccinimide ester (NSU). The NSU group reacts readily with the amino residue of the peptide. In the synthesis of oligomers a lipophilic portion is connected to a hydrophilic portion by ether linkage. The lipophilic portion is preferably selected from among alkyl, cholesterol and adamantyl moieties. Polyethylene glycol having free hydroxyl group at one end and carboxylic group at the other end is selected. This group of acidic oligomers can be coupled to peptide drugs by first reacting the carboxylic group with NSU. A solution of the activated oligomers in inert solvent is then treated with the desired peptide drug dissolved in a suitable solvent. Still additionally U.S. Pat. No. 6,624,245 discloses that biologically active agents may be incorporated into crosslinked synthetic polymer compositions of this patent by admixture, or by binding these agents to functional groups on the synthetic polymers. The crosslinked polymers form a biocompatible gel, prepared by a method that includes: (a) forming a reactive composition by admixing a biocompatible crosslinking component A having m sulfhydryl groups with a biocompatible crosslinking component B having n sulfhydryl-reactive groups that are capable of covalent reaction with each other upon admixture of components A and B; thereby form a gel.

In a further example, U.S. Pat. No. 5,936,035 discloses that biologically active agents may be incorporated into collagen employed in bioadhesives described in this patent by admixture. Alternatively, the agents may be covalently linked to the collagen using a crosslinking agent such as a functionally activated polyethylene glycol, or affinity bound to the collagen using a binding ligand. The bioadhesives are prepared, for example, from collagen and a multifunctionally activated synthetic hydrophilic polymer that are mixed to initiate crosslinking. Still additionally, U.S. Pat. No. 5,702,717 discloses a delivery liquid as an aqueous solution having dissolved or dispersed therein a drug intimately contained in a biodegradable block copolymer matrix. The copolymer has a reverse gelation temperature below the body temperature of a subject. The liquid is parenterally administered into the subject with the liquid forming a gel depot of the drug and block polymer as the temperature of the liquid is raised by the body temperature of the animal above the reverse gelation temperature of the block copolymer.

Yet further U.S. Pat. No. 6,201,072 discloses mixing a biodegradable ABA- or BAB-type tri-block polymer copolymer and peptide/protein drugs, and/or other types of drugs, to prepare an aqueous solution of the copolymer below the gelation temperature to form a drug delivery liquid where the drug may be either partially or completely dissolved. When the drug is partially dissolved, or when the drug is essentially insoluble, the drug exists in a colloidal state such as a suspension or emulsion. When administered to a patient, this drug delivery liquid undergoes a reversible thermal gelation since body temperature will be above the gelation temperature. Still additionally U.S. Pat. No. 6,589,549 discloses use of known microencapsulation techniques to incorporate bio-active agents into microparticle carriers. Commonly employed methods include: (a) phase separation and subsequent organic solvent evaporation (include O/W emulsion, W/O emulsions, O/O′ emulsions and W/O/W emulsions), (b) coacervation, (c) melt dispersion; (d) spray drying, (e) spray congealing, (f) air suspension coating; and (h) pan coating. Furthermore U.S. Patent Application Publications 20010009662 and 20040156819 disclose the synthesis of block copolymers by first synthesizing an ABA triblock or AB diblock. In this general reaction, a pre-prepared poly(oxyalkylene) B block is preferably reacted with a hydroxyacid or its cyclic dimer to produce the low molecular weight ABA triblock or AB diblock. Once the ABA triblock or AB diblock is formed, the hydroxyl groups at the end(s) of the molecule are reacted with difunctional chain extenders or couplers, for example, diisocyanates. This reaction produces a chain extended polymer which is readily used to prepare films and various related structures, gels, dispersions, suspensions, and viscous solutions. Bioactive agents may be delivered pursuant to methods of the publication.

Also, U.S. Pat. No. 6,870,012 discloses producing Aₙ(BCB)Aₙ pentablocks wherein a BCB triblock is a hydroxyacid, cyclic dimer or a related monomer to produce a pentameric Aₙ(BCB)Aₙ pentablock. Once the pentamer is formed, it is reacted with a chain-extender, E, to produce a chain-extended Aₙ(BCB)Aₙ pentablock of structure {[Aₙ(BCB)Aₙ]E}ₘ. Alternatively, the pentameric polymer may be reacted with crosslinking agent to produce a crosslinked polymeric system. Bioactive agents may be delivered pursuant to this patent. Further, U.S. Pat. No. 5,591,709 discloses combining a delivery polymer including, for example, numerous hydrogels in hydrated or unhydrated form, such as those derived from hydroxyethylmethacrylate (HEMA), glycerolmethacrylate (GMA) and polyvinylpyrrolidone (PVP), polyethylene glycol (PEG), various carbohydrates, cellulose and related hydrophilic cellulose polymers, dextran, polyethyleneoxide, dextran-polyethylene, acrylamide, polyacrylamide, amylose, collagen, gelatin, sepharose, agarose (for example, as an agarose saturated gel), related polymers and mixtures thereof, with a hormone or growth factor to promote wound healing.

U.S. Pat. No. 6,833,408 discloses a simple method for incorporating biologically active agents containing nucleophilic groups into a crosslinked polymer composition described in this patent involves mixing the active agent with a polyelectrophilic component prior to addition of the polynucleophilic component. A large variety of synthetic and natural polymers are described in this patent that may be used in this method. Additionally U.S. Pat. No. 6,624,245 discloses that biologically active agents may be incorporated into crosslinked synthetic polymer composition described in the patent by admixture or by binding these agents to functional groups on the synthetic polymers. The reactive composition used to form the crosslinked polymer includes at least two crosslinkable components: a first component which has m sulfhydryl groups, and a second component which has n sulfhydryl-reactive groups.

U.S. Pat. No. 6,534,591 discloses a general method for preparing a composition for the delivery of a negatively charged compound (such as a protein or drug). A first synthetic polymer containing two or more nucleophilic groups is reacted with a second synthetic polymer containing two or more electrophilic group. Biologically active agents may be incorporated into the crosslinked synthetic polymer composition by admixture or by binding these agents with the functional groups on the synthetic polymers.

Additionally U.S. Pat. No. 5,019,400 describes a process for preparing microspheres using cryogenic temperatures to freeze polymer-biologically active agent mixtures into polymeric microspheres with very high retention of biological activity and material. A cryogenic process for extracting solvent from a solution of a polymer including suspended particles of an active agent results in microspheres containing the active agent. Further, U.S. Pat. No. 5,143,661 discloses a process for preparing compositions comprising microcapsules by phase separation microencapsulation wherein the hardening agent employed is a volatile silicone fluid, wherein the microcapsules include a pharmaceutical agent. Still further, U.S. Pat. No. 6,117,455 discloses a sustained-release microcapsule which is obtained by selecting a dispersion of an amorphous water-soluble pharmaceutical agent in a solution of a polymer in an organic solvent; dispersing the dispersion of amorphous water-soluble pharmaceutical agent in an aqueous phase to prepare an s/o/w emulsion; and subjecting the s/o/w emulsion to in-water drying.

Further still U.S. Pat. No. 5,417,982 discloses a process for making a controlled release formulation comprising microspheres of a drug or hormone suspended in a biodegradable polymer matrix polymer, said process including the steps of a) preparing an aqueous solution of at least two highly water soluble biodegradable polymers and adding thereto an active ingredient of a hormone or drug, b) mixing the solution and active ingredient with an emulsifying medium to form a homogenized microdroplet suspension, c) adding the homogenized microdroplet suspension slowly to a first organic solvent which contains a small concentration of a first surfactant, while stirring the microdroplet suspension and solvent, thus causing microspheres to precipitate, d) separating the microspheres from the first solvent and adding a solution of a (d, 1 lactide-glycolide) copolymer in a second organic solvent which contains a small concentration of a second surfactant, and e) slowly evaporating the solvent, leaving behind coated microspheres.

Still additionally U.S. Pat. No. 5,718,921 discloses a method for preparation of multiwalled biodegradable polymeric drug delivery devices using relatively low temperatures and non-aqueous solutions which is particularly useful with polyanhydrides, and thermolabile drugs. In a first embodiment, the polymer is dissolved in a volatile organic solvent, the drug is dispersed or dissolved in the polymer solution, the mixture is suspended in an organic oil, and the organic solvent is extracted into the oil, creating microspheres. In a second embodiment, the polymer is dissolved in organic solvent with or without the drug, and the mixture is suspended in glycerol. The suspension is frozen and the organic solvent slowly evaporated. Using these embodiments, multi-walled microspheres having each wall degrading at a different rate or containing different drugs can be manufactured. Further yet U.S. Pat. No. 5,731,005 discloses several method for forming hydrogel microspheres of a protein and a second polymer having groups oppositely charged, and containing a drug.

Further still U.S. Pat. No. 6,306,406 discloses a process for preparing a drug delivery system for controlled release of calcitonin, the process including: (a) dissolving calcitonin and a hydrophobic biodegradable polymer selected from the group consisting of polyglycolic acid, polylactic acid, copolymers of glycolic acid and L- or D,L-lactic acid, and copolymers of glycolide and L- or D,L-lactide, in a first solvent and dispersing the solution in a second solvent to form microdroplets, (b) extracting the first solvent from the microdroplets, causing the calcitonin to adsorb to the polymer, and hardening the polymer such that calcitonin-loaded microspheres are formed. Additionally U.S. Pat. No. 6,517,859 discloses the preparation of dopamine microspheres. A solution of 50:50 poly(DL-lactide-co-glycolide) (“DL-PLG”) in dichloromethane was prepared. Dopamine (3-hydroxytyramine hydrochloride) is suspended in the polymer solution. Silicone oil is added, then the mixture was poured into heptane, forming dopamine microspheres, which are collected and dried.

The patents and patent application publications cited above are incorporated by reference herein in their entireties.

A sustained release composition or medicament may be prepared by conjugating any one or more pharmaceutically active agents disclosed herein, or a precursor thereof that gives rise to the pharmaceutically active agent in situ, to a polymer component of a sustained release formulation.

Any equivalent process for producing a sustained release pharmaceutical composition including one or both of a peptide ligand that binds a zeta receptor and at least one cancer chemotherapeutic agent, known to a worker of skill in fields such as pharmaceutical chemistry, drug delivery, and similar fields related to the field of the present invention, is encompassed within the scope of the present invention. Such a process provides a pharmaceutical composition including one or both of a peptide ligand that binds a zeta receptor and at least one cancer chemotherapeutic agent, and a sustained release formulation. The resulting pharmaceutical composition releases one or both of a peptide ligand of a zeta receptor, or the one or more cancer chemotherapeutic agents, in 1 day or greater, or about 2 days or greater, or about 4 days or greater, or about 1 week or greater, or about 10 days or greater, or about 2 weeks or greater, or about 20 days or greater, or about 3 weeks or greater, or about 4 weeks or greater, or about 5 weeks or greater, or about 6 weeks or greater, or about 7 weeks or greater, or about 8 weeks or greater, or about 9 weeks or greater, or about 10 weeks or greater, or about 11 weeks or greater, or about 12 weeks or greater, or about 13 weeks or greater, or even longer periods than any of the above.

Combinations

The present invention provides a combination including at least a portion of a tissue comprising one or more cells harboring a zeta receptor, in contact with a pharmaceutical composition comprising

a) one or both of
i) a peptide ligand that binds a zeta receptor and
ii) at least one cancer chemotherapeutic agent, and

b) a sustained release formulation.

The pharmaceutical composition and at least the portion of the tissue having cells harboring a zeta receptor are physically juxtaposed with respect to each other. In certain cases it is possible that an intervening biological structure such as a membrane, a tendon, a cartilage or a similar anatomical structure is interposed between a tissue and a juxtaposed medicament. Because of the intimate contact between the tissue and the medicament, or their close proximity, an active agent released from the medicament has a minimal diffusion path to reach a cell containing a target of an active agent contained in the medicament. This facilitates the elicitation of a characteristic growth inhibitory response. Pharmacologically, it may be understood that, because 1) an active agent has a high affinity for its target substance (i.e., exhibits a low Kd for binding thereto), and 2) the intimate or close proximity between a medicament in a combination and the tissue containing the target provides relatively high local concentration of the active agent in the immediate vicinity of the target, a medicament may be formulated to contain relatively low amounts of an active agent. That is, only sufficient active agent need be included in the medicament to ensure maintenance of an effective local concentration of the active agent in the proximity of the target to provide a predetermined amount, such as a given multiple of the Kd (dissociation constant) or ED50 (50% effective dose concentration) value, to treat the target tissue throughout substantially the entire period of sustained release. This feature is a highly advantageous property of the combinations and methods of the invention.

In various embodiments of the combinations and methods of the invention the medicament is in contact with a tumor margin, or the medicament is applied to a tumor margin. As used herein, “margin”, “tumor margin” and similar terms and phrases relate to a tissue or region surrounding a tumor or a cancer in which the stroma or other normal, healthy tissue that may have been altered by the presence of the tumor or cancer. More particularly, these terms relate to the edges, borders, or boundaries of tumor or cancer. For example, a tumor margin can include tumor cells that have grown beyond the visibly discernible edge of the tumor and can also include stromal regions that have been altered due to the presence of the tumor. In the case of ablation of a tumor or cancer, the margin includes tissues that usually appear to be normal to the naked eye that are removed along with the discernible tumor or cancer. The margin can generally extend from about 0.2 cm to about 3 cm, or even further, from a primary tumor or cancer, depending upon the size of the primary tumor or cancer. The margin becomes accessible as the result of a surgical procedure which removes a preponderance of the mass of a tumor or cancer from a subject. As indicated, the tumor margin may include one or more cells that are a cancer cell or a precancerous cell remaining in the subject.

In various embodiments of the combinations of the invention, a composition, pharmaceutical composition, or medicament of the invention includes a) one or both of a peptide ligand that binds a zeta receptor and at least one cancer chemotherapeutic agent; and b) a sustained release formulation. In certain embodiments of the combinations the medicament includes a peptide ligand of a zeta receptor, which may be an enkephalin, a fragment of an enkephalin, or a derivative thereof. In additional embodiments the medicament includes a zeta receptor ligand that may be an endorphin, a fragment of an endorphin, or a derivative thereof. In still further embodiments the medicament includes a zeta receptor ligand that may be a dynorphin, a fragment of a dynorphin, or a derivative thereof. In yet additional embodiments the medicament includes one or more cancer chemotherapeutic agents, such as, by way of nonlimiting example, the cancer chemotherapeutic agents disclosed herein.

In still additional embodiments of the combinations the medicament includes both a peptide ligand of a zeta receptor and at least one cancer chemotherapeutic agent. The peptide component may be an enkephalin, a fragment of an enkephalin, or a derivative thereof; or an endorphin, a fragment of an endorphin, or a derivative thereof; or a dynorphin, a fragment of a dynorphin, or a derivative thereof.

The one or more cancer chemotherapeutic agents in the medicament of the combination may be chosen, by way of nonlimiting example, from among asparaginase, hydroxyurea, altretamine, bleomycin, dactinomycin, doxorubicin, etoposide, teniposide, plicamydin, nitrogen mustards such as chlorambucil, cyclophosphamide, isofamide, mechlorethamine, melphalan, uracil mustard; an aziridine such as thiotepa, methanesulphonate esters such as busulfan, nitroso ureas such as carmustine, lomustine, streptozocin; platinum complexes, such as cisplatin and carboplatin; mitomycin, procarbazine, dacarbazine; amsacrine, daunorubicin, adriamycin, idarubicin, mitoxantrone; etoposide; teniposide, folate antagonists such as methotrexate and trimetrexate; pyrimidine antagonists, such as fluorouracil, fluorodeoxyuridine, CB3717, azacytidine, cytarabine, and floxuridine; purine antagonists such as mercaptopurine, 6-thioguanine, fludarabine, pentostatin, vincristine, vinblastine, paclitaxel, estrogens, conjugated estrogens, ethinyl estradiol and diethylstilbesterol, chlortrianisen and idenestrol; progestins such as hydroxyprogesterone caproate, medroxyprogesterone, and megestrol; androgens such as testosterone, testosterone propionate; fluoxymesterone, and methyltestosterone, prednisone, dexamethasone, methylprednisolone, prednisolone, leuprolide acetate, goserelin acetate, tamoxifen, flutamide; mitotane; and aminoglutethimide.

Nonlimiting examples of monoclonal antibody cancer therapeutic agents that may be used in the medicaments of the invention include Rituximab (Rituxan), Trastuzumab (Herceptin), Alemtuzumab (Campath), Cetuximab (Erbitux), Bevacizumab (Avastin), Ibritumomab (Zevalin), or Tositumomab, or two or more of them.

The sustained release formulation included in the medicament forming a combination may be any of a wide range of formulations. In general, sustained release formulations employed in the compositions, combinations and methods of the present invention are biocompatible, and in many embodiments are biodegradable. Nonlimiting examples of such formulations include gels, hydrogels, formulations applied by spray, bioadhesive formulations, and similar formulations, certain examples of which have been described hereinabove. Thus, by way of nonlimiting example, a sustained release formulation may include a natural polymer, such as a protein, a polysaccharide, a mucopolysaccharide, and the like. A protein component may be an albumin, a fibrinogen or a fibrin or solubilized derivative thereof, any of the several types of collagen in both fibril form or disaggregated, any of the several types of collagen that are atelocollagens, any of the several types of collagen that have been partially degraded in molecular weight, various gelatins derived from the several types of collagen, and the like. Polysaccharides include cellulose, cellulose derivatives such as alkyl celluloses, carboxyalkyl celluloses, glycogen, biocompatible vegetable gums, chitin, chitosan, biocompatible seaweed gums, chondroitin sulfate, hyaluronic acid, and the like. Nonlimiting examples of sustained release formulations that may be used in the combinations of the present invention include those disclosed in patents and patent application publications cited herein, and incorporated by reference herein in their entireties.

Further, by way of nonlimiting example, a sustained release formulation may include a synthetic polymer, such as a homopolymers or a copolymer, wherein the copolymer may be a homogeneously copolymerized copolymer or a block copolymer. Components of synthetic polymers may be chosen, by way of nonlimiting example, from among poly(hydroxyacyl)esters; poly(lactone)esters; various polyvinyl derivatives including polyvinylpyrrolidone, polyvinyl alcohol, and the like; polyethyleneoxide and homologous polyalkyleneoxides, both capped and uncapped; polycarbonates; polyanhydrides; polyamines; polyurethanes; polyesteramides; polyorthoesters; polydioxanones; polyacetals, polyketals; polycarbonates; polyorthocarbonates; polyphosphazenes; succinates; poly(malic acid); and poly(amino acids).

A sustained release formulation used in a medicament forming a combination may be comprised of a single polymer, or a combination or mixture of more than one polymer.

A polymer or copolymer chosen for use in a medicament to form a combination is to be biocompatible. In many embodiments the polymer additionally is biodegradable. A polymer used in a medicament may be formulated in an aqueous medium, or a dispersing solvent may include biocompatible nonaqueous solvents or cosolvents according to the properties of the chosen polymer, to permit the composition to be employed as a gel, a hydrogel, a formulation applied by spray, a bioadhesive formulation, or a similar formulation.

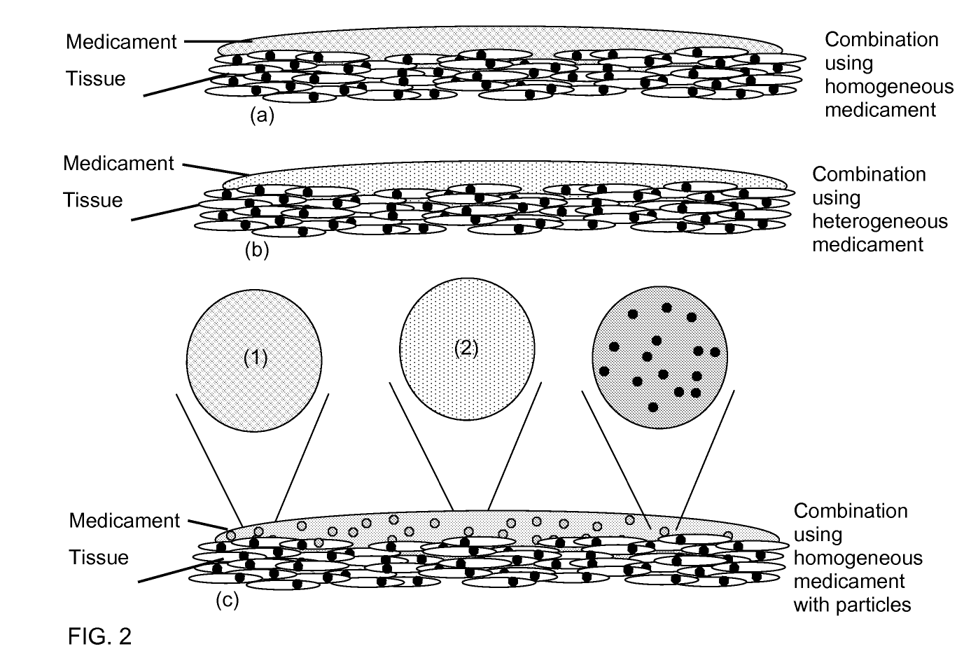

The one or more pharmaceutical active agents included in the pharmaceutical composition forming a combination may be incorporated as a solution, a dispersion, a suspension, a comminution, or any similar composition, according to the respective properties of the active agent and the sustained release formulation. A schematic representation of certain embodiments of a combination of the invention are shown in FIG. 2. In these representations a pharmaceutical composition of the invention is disposed upon a tissue that includes cells harboring a zeta receptor. A combination in which an active agent is incorporated in a pharmaceutical composition as a solution is shown in FIG. 2 panel (a); and a combination in which an active agent is incorporated as a dispersion, a suspension, or a comminution is shown in FIG. 2, panel (b).

Additionally, sustained release formulations present in a combination may be constituted of, or may include as one of several components, particles, beads, microparticles, nanoparticles, and similar formed entities that include a pharmaceutically active agent as a component. A schematic representation of embodiments of a combination of the invention, in which a pharmaceutical composition includes a particle, microparticle or nanoparticle is shown in FIG. 2, panel (c). In panel (c), a particle in which an active agent is incorporated as a solution is shown in the blowup of a particle in (1). A particle in which an active agent is included as a dispersion, a suspension, or a comminution of relatively small insoluble solids is shown in the blowup of (2), and an example in which an agent is included in a particle as a relatively large insoluble solid is shown in the blowup of (3). The representations in FIG. 2 are nonlimiting; combinations in which alternative or equivalent designs of pharmaceutical compositions are employed are also included within the scope of the present invention.

Furthermore, a sustained release composition or medicament forming a combination may contain any of the pharmaceutically active agents disclosed herein, or a precursor thereof that gives rise to the pharmaceutically active agent in situ, conjugated to a polymer component of a sustained release formulation.

In general, a composition, or a pharmaceutical composition, or a medicament, forming a combination releases the pharmaceutically active agent over a period of time such that the length of time over which the active agent is bioavailable is greater than it would be in the absence of the formulation. Thus the bioavailability may endure 1 day or longer, or about 2 days or longer, or about 4 days or longer, or about 1 week or longer, or about 10 days or longer, or about 2 weeks or longer, or about 20 days or longer, or about 3 weeks or longer, or about 4 weeks or longer, or about 5 weeks or longer, or about 6 weeks or longer, or about 7 weeks or longer, or about 8 weeks or longer, or about 9 weeks or longer, or about 10 weeks or longer, or about 11 weeks or longer, or about 12 weeks or longer, or about 13 weeks or longer, or even longer periods than any of the above.

A medicament forming a combination may furthermore be composed of more than one composition, each of which includes one or both of a peptide ligand that binds a zeta receptor and at least one cancer chemotherapeutic agent, and a sustained release formulation that differs from the others. By way of nonlimiting example, a first pharmaceutical composition including one or both of a first peptide ligand that binds a zeta receptor and at least one first cancer chemotherapeutic agent, and a first sustained release formulation, may be combined with a second pharmaceutical composition including one or both of a second peptide ligand that binds a zeta receptor and at least one second cancer chemotherapeutic agent, and a second sustained release formulation. The various component pharmaceutical compositions may include different active agents, and may be characterized by having different sustained release formulations whose release kinetics may be the same, or may that may be similar, or that differ substantially from each other. Thus, by way of nonlimiting example, a gel composition including a peptide ligand of a zeta receptor and having relatively rapid release kinetics, may be combined or admixed with a microsphere composition including one or more cancer chemotherapeutic agents and having relatively slow release kinetics. Alternatively, as another example, a gel composition including a first set of cancer chemotherapeutic agents and having relatively rapid release kinetics may be combined with a microcapsule including a second set of cancer chemotherapeutic agents having relatively slow release kinetics. Use of combined medicaments as described above permits addressing different molecular targets within a cancer cell or a precancerous cell, thereby increasing the likelihood of inducing death of the cancer cell and/or the precancerous cell, and optimizing the likelihood of remission of the cancer. Any of the component compositions in the combined medicament described above may release its pharmaceutical active agent in 1 day or longer, or about 2 days or longer, or about 4 days or longer, or about 1 week or longer, or about 10 days or longer, or about 2 weeks or longer, or about 20 days or longer, or about 3 weeks or longer, or about 4 weeks or longer, or about 5 weeks or longer, or about 6 weeks or longer, or about 7 weeks or longer, or about 8 weeks or longer, or about 9 weeks or longer, or about 10 weeks or longer, or about 11 weeks or longer, or about 12 weeks or longer, or about 13 weeks or longer, or even longer periods than any of the above.

Method of Forming a Combination

The present invention provides a method of forming a combination that includes a step of disposing a pharmaceutical composition upon at least a portion of a tissue that contains one or more cells harboring a zeta receptor, wherein the pharmaceutical composition comprises

a) one or both of
i) a peptide ligand that binds a zeta receptor and
ii) at least one cancer chemotherapeutic agent; and

b) a sustained release formulation.

In methods of forming a combination the disposing may be carried out by coating, spreading, spraying, dispersing, flowing, applying, or generally bringing the pharmaceutical composition into intimate contact with the tissue. In significant embodiments substantially an entire cavity exposing such a tissue is so contacted.

In various embodiments of methods of forming a combination, the pharmaceutical composition may include a peptide ligand that binds a zeta receptor, or the pharmaceutical composition may include at least one cancer chemotherapeutic agent., or the pharmaceutical composition may include both a peptide ligand that binds a zeta receptor and at least one cancer chemotherapeutic agent. In various additional embodiments of this method the peptide ligand includes an enkephalin or a derivative thereof, or an endorphin or a derivative thereof.

In still other various embodiments of methods of forming a combination the one or more cells is a cancer cell or a precancerous cell, and in certain various embodiments the one or more cells are located in a subject. In additional various embodiments of this method the one or more cells are at a margin of a tumor wherein the margin is exposed by removal of a preponderance of the tumor.

In various additional embodiments of methods of forming a combination the sustained release formulation is biocompatible. In further embodiments of this method release of the peptide ligand, or the one or more cancer chemotherapeutic agents, or both, contained therein from the pharmaceutical composition occurs in a substantially therapeutically effective amount over a time period ranging from about 1 day to about 13 weeks. In yet additional various embodiments of this method, the sustained release formulation includes a gel, a hydrogel, a bioadhesive, a crosslinked gel, a microsphere, a microparticle, or a nanoparticle, or a combination of any of them. Still further, in various embodiments of this method the composition includes

a) one or both of
i) a peptide ligand that binds a zeta receptor and
ii) at least one cancer chemotherapeutic agent; conjugated to

b) a polymeric carrier.

The tissue containing one or more cells harboring a zeta receptor may be any tissue of any mammal, including a human.

Method of Treating a Pathology

The present invention additionally provides a method of treating a pathology that responds to a peptide ligand that binds a zeta receptor. The method includes a step of contacting at least a portion of a tissue that is characteristic of the pathology and that contains one or more cells harboring a zeta receptor, with a pharmaceutical composition that includes

a) one or both of
i) a peptide ligand that binds a zeta receptor and
ii) at least one cancer chemotherapeutic agent, in amounts effective to treat the pathology; and

b) a sustained release formulation.

In various embodiments of this method the pathology is a cancer, and in further various embodiments of this method the one or more cells is a cancer cell or a precancerous cell. A cancer that may be treated includes, by way nonlimiting example, acidophil adenoma, acoustic neuroma, adenocarcinoma, adenolymphoma, adenoma, adrenal gland tumor, ameloblastoma, anaplastic carcinoma of the thyroid, angiofibroma, angioma, angiosarcoma, apudoma, arrhenoblastoma, astroblastoma, astrocytoma, atrial myxoma, basal cell carcinoma, bone cancer, bone tumor, brainstem glioma, brain tumor, breast cancer, Burkitt's lymphoma, carcinoma, cerebellar astrocytoma, cervical cancer, cherry angioma, cholangiocarcinoma, cholangioma, chondroblastoma, chondroma, chondrosarcoma, chorioblastoma, choriocarcinoma, CNS leukaemia, colon cancer, colorectal cancer, common acute lymphoblastic leukaemia, connective tumor, corticotrophic adenoma, craniopharyngioma, cutaneous T-cell lymphoma, cystocarcinoma, cystofibroma, cystoma, cytoma, ductal carcinoma in situ, ductal papilloma, dysgerminoma, encephaloma, endometrial carcinoma, endothelioma, eosinophilic granuloma, ependymoma, epithelioma, erythroleukemia, Ewing's sarcoma, extra nodal lymphoma, fibroadenoma, fibroma, fibromyoma, fibrosarcoma, follicular cancer of the thyroid, ganglioglioma, gastrinoma, giant cell granuloma, glioblastoma multiforme, glioma, gonadoblastoma, haemangioblastoma, haemangioendothelioblastoma, haemangioendothelioma, haemangiopericytoma, haematolymphangioma, haemocytoblastoma, haemocytoma, hairy cell leukaemia, hamartoma, hepatocarcinoma, hepatocellular carcinoma, hepatoma, histoma, Hodgkin's disease, hypernephroma, infiltrating ductal cell carcinoma, insulinoma, juvenile angiofibroma, Kaposi sarcoma, kidney tumor, large cell lymphoma, lipoma, liver cancer, liver metastases, Lucke carcinoma, lung cancer, lymphadenoma, lymphangioma, lymphocytic leukaemia, lymphocytic lymphoma, lymphocytoma, lymphoedema, lymphoma, malignant melanoma, malignant mesothelioma, malignant teratoma, mastocytoma, medulloblastoma, melanoma, meningioma, mesothelioma, Morton's neuroma, multiple myeloma, myeloblastoma, myelolipoma, myeloma, myoblastoma, myofibroma, myolipoma, myoma, myxoma, nasopharyngeal carcinoma, nephroblastoma, neuroblastoma, neurofibroma, neurofibromatosis, neuroglioma, neuroma, non-Hodgkin's lymphoma, nonmelanoma skin cancer, oligodendroglioma, optic glioma, osteochondroma, osteogenic sarcoma, osteoma, osteosarcoma, ovarian cancer, pancreatic cancer, papilloma, plasmacytoma, primary brain tumor, progonoma, prolactinoma, prostate cancer, renal cell carcinoma, retinoblastoma, rhabdomyosarcoma, rhabdosarcoma, sarcoma, seminoma, skin cancer, small cell carcinoma, squamous cell carcinoma, strawberry haemangioma, teratoma, testicular cancer, thymoma, thyroid cancer, vascular neoplasms, vestibular schwannoma, Wilm's tumor, and Yaba tumor.

Additionally in various embodiments of methods of treating a pathology the one or more cells are located in a subject. In additional various embodiments of methods of treating a pathology the one or more cells are at a margin of a tumor wherein the margin is exposed by removal of a preponderance of the tumor.

In methods of treating a pathology the contacting step provides that the pharmaceutical composition and at least the portion of the tissue having cells harboring a zeta receptor are physically juxtaposed with respect to each other. The contacting may be carried out by coating, spreading, spraying, dispersing, flowing, applying, or generally bringing the medicament into intimate contact with the tissue. In significant embodiments substantially an entire cavity exposing such a tissue is so contacted. An active agent released from the medicament so juxtaposed has a minimal diffusion path to reach a cell containing a target of an active agent contained in the medicament. This facilitates elicitation of a characteristic growth inhibitory response.

In various embodiments of methods of treating a pathology the medicament is applied to a tumor margin. In various embodiments of methods of treating a pathology, a composition, pharmaceutical composition, or medicament of the invention includes a) one or both of a peptide ligand that binds a zeta receptor and at least one cancer chemotherapeutic agent; and b) a sustained release formulation. In certain embodiments of methods of treating a pathology, the medicament includes a peptide ligand of a zeta receptor that may be an enkephalin, a fragment of an enkephalin, or a derivative thereof, or an endorphin, a fragment of an endorphin, or a derivative thereof, or a dynorphin, a fragment of a dynorphin, or a derivative thereof. In yet additional embodiments the medicament includes one or more cancer chemotherapeutic agents.

In still additional embodiments of the combinations the medicament includes both a peptide ligand of a zeta receptor and at least one cancer chemotherapeutic agent. The peptide component may be an enkephalin, a fragment of an enkephalin, or a derivative thereof; or an endorphin, a fragment of an endorphin, or a derivative thereof; or a dynorphin, a fragment of a dynorphin, or a derivative thereof.

The one or more cancer chemotherapeutic agents in the medicament employed in methods of treating a pathology may be chosen, by way of nonlimiting example, from among asparaginase, hydroxyurea, altretamine, bleomycin, dactinomycin, doxorubicin, etoposide, teniposide, plicamydin, nitrogen mustards such as chlorambucil, cyclophosphamide, isofamide, mechlorethamine, melphalan, uracil mustard; an aziridine such as thiotepa, methanesulphonate esters such as busulfan, nitroso ureas such as carmustine, lomustine, streptozocin; platinum complexes, such as cisplatin and carboplatin; mitomycin, procarbazine, dacarbazine; amsacrine, daunorubicin, adriamycin, idarubicin, mitoxantrone; etoposide; teniposide, folate antagonists such as methotrexate and trimetrexate; pyrimidine antagonists, such as fluorouracil, fluorodeoxyuridine, CB3717, azacytidine, cytarabine, and floxuridine; purine antagonists such as mercaptopurine, 6-thioguanine, fludarabine, pentostatin, vincristine, vinblastine, paclitaxel, estrogens, conjugated estrogens, ethinyl estradiol and diethylstilbesterol, chlortrianisen and idenestrol; progestins such as hydroxyprogesterone caproate, medroxyprogesterone, and megestrol; androgens such as testosterone, testosterone propionate; fluoxymesterone, and methyltestosterone, prednisone, dexamethasone, methylprednisolone, prednisolone, leuprolide acetate, goserelin acetate, tamoxifen, flutamide; mitotane; and aminoglutethimide.

Nonlimiting examples of monoclonal antibody cancer therapeutic agents that may be used in the medicaments of the invention include Rituximab (Rituxan), Trastuzumab (Herceptin), Alemtuzumab (Campath), Cetuximab (Erbitux), Bevacizumab (Avastin), Ibritumomab (Zevalin), or Tositumomab, or two or more of them.

The sustained release formulation included in the medicament that is used to contact the tissue may be any of a wide range of formulations. In general, sustained release formulations employed in the compositions, combinations and methods of the present invention are biocompatible, and in many embodiments are biodegradable. Nonlimiting examples of such formulations include gels, hydrogels, formulations applied by spray, bioadhesive formulations, and similar formulations, certain examples of which have been described hereinabove. Thus, by way of nonlimiting example, a sustained release formulation may include a natural polymer, such as a protein, a polysaccharide, a mucopolysaccharide, and the like. A protein component may be an albumin, a fibrinogen or a fibrin or solubilized derivative thereof, any of the several types of collagen in both fibril form or disaggregated, any of the several types of collagen that are atelocollagens, any of the several types of collagen that have been partially degraded in molecular weight, various gelatins derived from the several types of collagen, and the like. Polysaccharides include cellulose, cellulose derivatives such as alkyl celluloses, carboxyalkyl celluloses, glycogen, biocompatible vegetable gums, chitin, chitosan, biocompatible seaweed gums, chondroitin sulfate, hyaluronic acid, and the like.

Further, by way of nonlimiting example, a sustained release formulation may include a synthetic polymer, such as a homopolymers or a copolymer, wherein the copolymer may be a homogeneously copolymerized copolymer or a block copolymer. Components of synthetic polymers may be chosen, by way of nonlimiting example, from among poly(hydroxyacyl)esters; poly(lactone)esters; various polyvinyl derivatives including polyvinylpyrrolidone, polyvinyl alcohol, and the like; polyethyleneoxide and homologous polyalkyleneoxides, both capped and uncapped; polycarbonates; polyanhydrides; polyamines; polyurethanes; polyesteramides; polyorthoesters; polydioxanones; polyacetals, polyketals; polycarbonates; polyorthocarbonates; polyphosphazenes; succinates; poly(malic acid); and poly(amino acids).

A sustained release formulation used in a medicament that contacts a tissue may be comprised of a single polymer, or a combination or mixture of more than one polymer. A polymer or copolymer chosen for use is to be biocompatible. In many embodiments the polymer additionally is biodegradable. A polymer used in a medicament may be formulated in an aqueous medium, or a dispersing solvent may include biocompatible nonaqueous solvents or cosolvents according to the properties of the chosen polymer, to permit the composition to be employed as a gel, a hydrogel, a formulation applied by spray, a bioadhesive formulation, or a similar formulation.

The one or more pharmaceutical active agents included in the pharmaceutical composition used to contact the tissue, in the methods of treating a pathology, may be incorporated as a solution, a dispersion, a suspension, a comminution, or any similar composition, according to the respective properties of the active agent and the sustained release formulation.

Additionally, sustained release formulations employed to form a medicament used to contact a tissue may be constituted of, or may include as one of several components, particles, beads, microparticles, nanoparticles, and similar formed entities that include a pharmaceutically active agent as a component.

Furthermore, a sustained release composition or medicament used to contact a tissue may contain any of the pharmaceutically active agents disclosed herein, or a precursor thereof that gives rise to the pharmaceutically active agent in situ, conjugated to a polymer component of a sustained release formulation.

Nonlimiting examples of sustained release formulations that may be used in methods of treating a pathology of the present invention include those disclosed in patents and patent application publications cited herein, and incorporated by reference herein in their entireties.

In general, in further embodiments of methods of treating a pathology, release of the peptide ligand, or the one or more cancer chemotherapeutic agents, or both, from the pharmaceutical composition employed in the contacting step occurs in a substantially therapeutically effective amount over a time period ranging from about 1 day to about 13 weeks. Thus the bioavailability may endure 1 day or longer, or about 2 days or longer, or about 4 days or longer, or about 1 week or longer, or about 10 days or longer, or about 2 weeks or longer, or about 20 days or longer, or about 3 weeks or longer, or about 4 weeks or longer, or about 5 weeks or longer, or about 6 weeks or longer, or about 7 weeks or longer, or about 8 weeks or longer, or about 9 weeks or longer, or about 10 weeks or longer, or about 11 weeks or longer, or about 12 weeks or longer, or about 13 weeks or longer, or even longer periods than any of the above.

A medicament used to contact a tissue in methods of treating a pathology may furthermore be composed of more than one composition, each of which includes one or both of a peptide ligand that binds a zeta receptor and at least one cancer chemotherapeutic agent, and a sustained release formulation that differs from the others. By way of nonlimiting example, a first pharmaceutical composition including one or both of a first peptide ligand that binds a zeta receptor and at least one first cancer chemotherapeutic agent, and a first sustained release formulation, may be combined with a second pharmaceutical composition including one or both of a second peptide ligand that binds a zeta receptor and at least one second cancer chemotherapeutic agent, and a second sustained release formulation. The various component pharmaceutical compositions may include different active agents, and may be characterized by having different sustained release formulations whose release kinetics may be the same, or may that may be similar, or that differ substantially from each other. Any of the component compositions in the combined medicament described above may release its pharmaceutical active agent in 1 day or greater, or about 2 days or greater, or about 4 days or greater, or about 1 week or greater, or about 10 days or greater, or about 2 weeks or greater, or about 20 days or greater, or about 3 weeks or greater, or about 4 weeks or greater, or about 5 weeks or greater, or about 6 weeks or greater, or about 7 weeks or greater, or about 8 weeks or greater, or about 9 weeks or greater, or about 10 weeks or greater, or about 11 weeks or greater, or about 12 weeks or greater, or about 13 weeks or greater, or even longer periods than any of the above.

In additional embodiments of methods of treating a pathology further treatment may include, in addition to the contacting step, systemic administration of a cancer chemotherapeutic agent. Such administration may be any of a variety of parenteral routes, including, by way of nonlimiting example, intravenous, subcutaneous, intramuscular, intraperitoneal, aerosol inhalation, buccal, vaginal, rectal, and the like. Further treatment may be administered on a single occasion, or it may be repeated according to a treatment regimen.

The tissue containing the one or more cells harboring a zeta receptor may be a tissue of any mammal, including a human. Treatment of the pathology provides inhibition of cancer cells that can generally be any inhibition of growth of the cancer cells as compared to the cancer cells without treatment. The inhibition is preferably at least about 10%, 20%, 30%, 40%, 50%, 60%, 70%, 80%, 90%, 95%, 96%, 97%, 98%, 99%, and ideally 100% inhibition of growth. The inhibition may be achieved by arrest of cell growth, by lysis of the cancer cells or by other means.

Method of Inhibiting Proliferation or Metastasis of a Cancer in a Subject

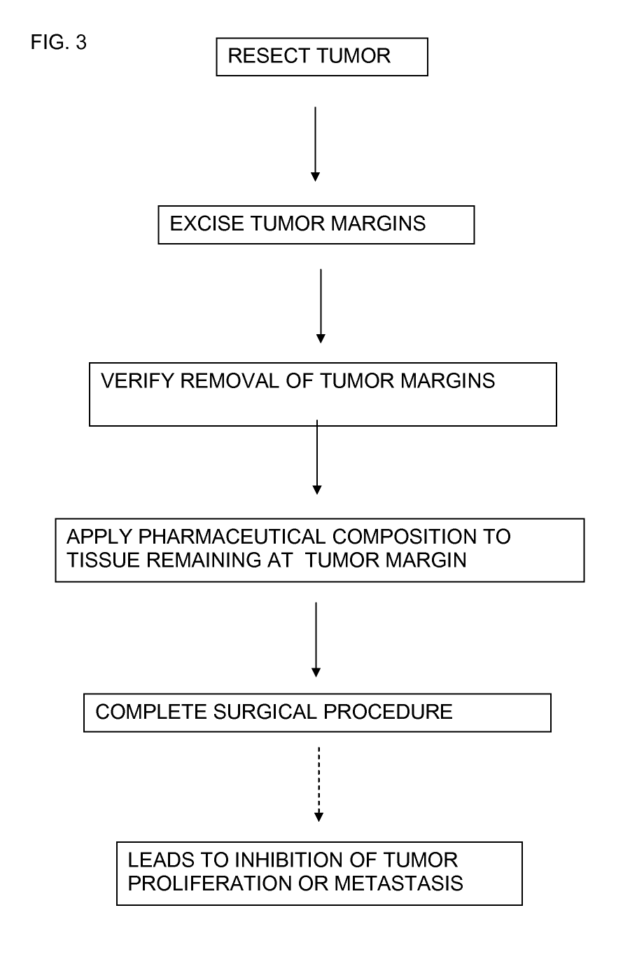

The invention additionally provides various methods of inhibiting proliferation or metastasis of a cancer in a subject. These methods include the steps of
a) surgically excising a tumor characterizing the cancer from the subject;
b) ensuring maximal removal of a tumor margin from the tumor site; and
c) disposing a medicament upon at least a portion of a tissue remaining at a tumor margin wherein the tissue contains one or more tumor cells (such as a cancer cell or a precancerous cell) harboring a zeta receptor. The medicament disposed on the tissue contains
   i) one or both of
      a′) a peptide ligand that binds a zeta receptor and
      b′) at least one cancer chemotherapeutic agent, in amounts effective to treat the tumor cells; and
   ii) a sustained release formulation.
      The steps of this method are effective to inhibit proliferation or metastasis of the cancer. A nonlimiting embodiment of a method of inhibiting proliferation or metastasis of a cancer in a subject is represented schematically in FIG. 3.

In various embodiments of methods of inhibiting proliferation or metastasis of a cancer, a cancer that may be treated includes, by way nonlimiting example, acidophil adenoma, acoustic neuroma, adenocarcinoma, adenolymphoma, adenoma, adrenal gland tumor, ameloblastoma, anaplastic carcinoma of the thyroid, angiofibroma, angioma, angiosarcoma, apudoma, arrhenoblastoma, astroblastoma, astrocytoma, atrial myxoma, basal cell carcinoma, bone cancer, bone tumor, brainstem glioma, brain tumor, breast cancer, Burkitt's lymphoma, carcinoma, cerebellar astrocytoma, cervical cancer, cherry angioma, cholangiocarcinoma, cholangioma, chondroblastoma, chondroma, chondrosarcoma, chorioblastoma, choriocarcinoma, CNS leukaemia, colon cancer, colorectal cancer, common acute lymphoblastic leukaemia, connective tumor, corticotrophic adenoma, craniopharyngioma, cutaneous T-cell lymphoma, cystocarcinoma, cystofibroma, cystoma, cytoma, ductal carcinoma in situ, ductal papilloma, dysgerminoma, encephaloma, endometrial carcinoma, endothelioma, eosinophilic granuloma, ependymoma, epithelioma, erythroleukemia, Ewing's sarcoma, extra nodal lymphoma, fibroadenoma, fibroma, fibromyoma, fibrosarcoma, follicular cancer of the thyroid, ganglioglioma, gastrinoma, giant cell granuloma, glioblastoma multiforme, glioma, gonadoblastoma, haemangioblastoma, haemangioendothelioblastoma, haemangioendothelioma, haemangiopericytoma, haematolymphangioma, haemocytoblastoma, haemocytoma, hairy cell leukaemia, hamartoma, hepatocarcinoma, hepatocellular carcinoma, hepatoma, histoma, Hodgkin's disease, hypernephroma, infiltrating ductal cell carcinoma, insulinoma, juvenile angiofibroma, Kaposi sarcoma, kidney tumor, large cell lymphoma, lipoma, liver cancer, liver metastases, Lucke carcinoma, lung cancer, lymphadenoma, lymphangioma, lymphocytic leukaemia, lymphocytic lymphoma, lymphocytoma, lymphoedema, lymphoma, malignant melanoma, malignant mesothelioma, malignant teratoma, mastocytoma, medulloblastoma, melanoma, meningioma, mesothelioma, Morton's neuroma, multiple myeloma, myeloblastoma, myelolipoma, myeloma, myoblastoma, myofibroma, myolipoma, myoma, myxoma, nasopharyngeal carcinoma, nephroblastoma, neuroblastoma, neurofibroma, neurofibromatosis, neuroglioma, neuroma, non-Hodgkin's lymphoma, nonmelanoma skin cancer, oligodendroglioma, optic glioma, osteochondroma, osteogenic sarcoma, osteoma, osteosarcoma, ovarian cancer, pancreatic cancer, papilloma, plasmacytoma, primary brain tumor, progonoma, prolactinoma, prostate cancer, renal cell carcinoma, retinoblastoma, rhabdomyosarcoma, rhabdosarcoma, sarcoma, seminoma, skin cancer, small cell carcinoma, squamous cell carcinoma, strawberry haemangioma, teratoma, testicular cancer, thymoma, thyroid cancer, vascular neoplasms, vestibular schwannoma, Wilm's tumor, and Yaba tumor.

Methods for excising a tumor from a subject are widely known among surgeons, oncologists, and other workers of skill in fields related to the present invention. By way of nonlimiting example, depending on the nature of the tumor and the organ affected, surgical resection of a tumor, such as a malignant tumor, is carried out by any of several methods known to workers of skill in fields related to the present invention. In one surgical procedure, an incision is made to open the region containing the tumor to view, and the tumor is removed by a surgeon. Alternatively, laparoscopy can be carried out by introducing a laparoscope through a small incision, for example under the guidance of a visible light or other imaging process to guide the placement and operation of the laparoscope. An additional procedure is use of a cryosurgical probe to excise a tumor by low-temperature freezing. The placement of the cryosurgical probe may be assisted by visible light or other imaging procedures. Still further, a tumor may be ablated by applying any of a variety of energy sources. Any equivalent procedure known to workers of skill in fields related to the present invention, and that is effective to excise or remove a tumor from a subject is also contemplated within the scope of the invention.

The therapeutic methods of the invention such as a method of inhibiting proliferation or metastasis of a cancer in a subject include a step of ensuring maximal removal of tumor margins from the tumor site. After excision of a malignant tumor, for example by surgical resection, or ablation of a malignant tumor by various methods, the remaining cavity has a high probability of having cancer cells and precancerous cells left at the site of the surgery. Residual cancer cells may arise from natural shedding of cancer cells from the tumor, as well as by being dislodged from a tumor mass during surgery. In addition, cells in tissue adjacent to the malignant cells of a cancerous tumor may differ from normal cells of the tissue because of their proximity to the cancer cells. Such cells may become predisposed to the subsequent transformation and development of a cancerous state; without wishing to be bound by theory it is possible that this occurs by virtue of various growth factors and/or metabolites secreted by the cancerous cells of the tumor. Residual cancer cells, since they may not be anchored in a tissue, are dangerous to the health of the patient because of their potential for metastasis and development of cancer at remote locations in the body, being disseminated into the systemic circulation and into the regional lymphatics. Precancerous cells are also dangerous to the patient because, when left in situ at the surgical site, may develop into cancer cells with the passage of time. Such secondary cancers may not become evident for months or years after a surgical resection procedure. For these reasons methods of the invention strive to maximize removal of tumor margins during surgical resection of a tumor.

A conventional way of determining the presence or absence of a tumor margin intraoperatively, known to workers of skill in fields related to the present invention, is to remove a sample of tissue from a region suspected of representing a remaining tumor margin, and subjecting the sample to pathological, or histopathological, examination. (Hermanek P et al., Semin Surg Oncol. 2000; 19(4):329-35). Depending on the pathology result, a surgeon may undertake to remove additional tissue from the suspect region prior to completing the procedure.

A variety of methods are known to workers of skill in fields related to the present invention for imaging tumors and identifying tumor margins. For example, according to U.S. Pat. No. 4,930,516, it was found that the shapes of the visible luminescence spectra from normal and cancerous tissue are substantially different. Thus, according to this patent, a method for detecting the presence of cancerous tissue involves illuminating a region to be examined with a beam of monochromatic light, and then comparing the resulting luminescence spectrum with the luminescence spectrum for a normal tissue, thereby determining the carcinomatoid status of the tissue in question.

U.S. Pat. Nos. 5,438,989, 5,699,798, 6,161,031, 6,241,672, and 6,671,540 disclose apparatuses and methods for delineating tumors and their margins. These patents state that a method for imaging a solid tumor involves administering a dye into an artery or vein perfusing the suspected tumor site in the area of interest. The dye may be characterized by absorption of light, or by fluorescence. More generally optical contrast enhancing agents may be used that enhance differences in the optical properties, or optical contrast, between normal and abnormal tissue. A video CCD of the inventive apparatus is focused upon the suspected solid tumor site (area of interest) and a high intensity light beam containing the wavelength absorbed by the dye illuminates the site. Images prior to and after dye administration are subtractively compared to produce difference images. U.S. Pat. No. 6,671,540 discloses general apparatuses and methods employing one or more light source(s) for illuminating an area of interest and one or more optical detector(s) capable of detecting and acquiring data relating to one or more optical properties of the area of interest.

According to U.S. Pat. No. 6,317,624 it was discovered that endogenous fluorescence at excitation wavelengths of about 340 to 400 nm is almost entirely suppressed within, and in the immediate vicinity of, basal and squamous cell carcinomas. Accordingly, the patent discloses a method of non-invasively detecting the margins of skin tumors by monitoring suppression of dermal fluorescence.

According to U.S. Pat. No. 6,375,634 tissue margins may be examined during a surgical procedure by pathology touch preparations or tumor bed cell washings for automated cell sorting obtained from the freshly exposed tissue walls after excision of a tumor. According to this patent, the tumor mass is first encapsulated by a composition prior to excision.

U.S. Pat. No. 6,377,841 and U.S. Patent Application Publication 20040044287 disclose that the intensity of a fluorescence peak from normal brain tissues is greater than that from primary brain tumorous tissues. In addition, diffuse reflectance (Rd) between 650 nm and 800 nm from white matter was significantly stronger than that from primary and secondary brain tumors. According to this patent the ratiometric comparison of these properties can be applied to identify tumor margins.

U.S. Patent Application Publication 20040152997 discloses a method and system for determining a condition of a selected region of tissue to facilitate the location of surgical resection margins. According to this publication electropotential and impedance are measured at one or more locations in an area of the body where tissue is to be removed, including use of an agent to enhance electrophysiological characteristics of that tissue. Differences in the profile are used to determine the borders between normal and abnormal tissue so as to facilitate what tissue to resect.

U.S. Patent Application Publication 20040181130 discloses another process to identify the margins of cancerous lesions. In a certain aspect, the publication proposes a method of visualizing the margins of a skin lesion. Generally, the method includes administering to a treatment area of the skin a low molecular weight immune response modulator (IRM) compound for a period of time and in an amount sufficient to permit visualization of the margins of a skin lesion in the treatment area.

Intraoperative infrared imaging of brain tumors has been shown to permit distinguishing between the tumor mass and the tumor margin by scanning local temperature during surgery (Gorbach A M et al., J. Neurosurg. 2004; 101:960-9). The differential vascularization of these zones contributes to thermal gradients and infrared emissions from them.

After it is ensured that as much of the tumor margin has been removed as is possible or practicable, at least a portion of the cavity exposed by the removal of the tumor is contacted with a medicament that includes a sustained release formulation. The contacting may be carried out by coating, spreading, spraying, dispersing, flowing, applying, or generally bringing the medicament into intimate contact with the tissue. In significant embodiments substantially the entire cavity is so contacted. The contacting of the cavity establishes intimate association between the residual tissue remaining at the peripheries of the cavity and the medicament, thus forming a tissue-medicament combination. The tissue which is coated with the medicament/pharmaceutical composition generally includes, or is suspected of including, cells from a tumor margin, which are thereby targets for the pharmaceutical active agent or agents contained within the pharmaceutical composition. The cells in the tissue may contact the composition directly; alternatively a tissue or organ structure such as a membraneous structure, a cartilaginous structure, a ligamental structure, or the like, may be inadvertently interposed between the tissue and the composition. The residual tumor margin may include cells that are cancer cells such as cancer cells derived from the body of the tumor as well as remaining in the margin, or the margin may include precancerous cells. In addition, the cavity created by the surgery may contain stray cancer cells from the body of the tumor that may have been dislodged from the tumor and remain in the cavity as free cells, not associated with a particular tissue structure.

In methods of inhibiting proliferation or metastasis of a cancer the disposing step provides that the pharmaceutical composition and at least the portion of the tissue having cells harboring a zeta receptor are physically juxtaposed with respect to each other. An active agent released from the medicament so juxtaposed has a minimal diffusion path to reach a cell containing a target of an active agent contained in the medicament. This facilitates elicitation of a characteristic growth inhibitory response.

In various embodiments of methods of inhibiting proliferation or metastasis of a cancer a composition, pharmaceutical composition, or medicament employed in the method includes a) one or both of a peptide ligand that binds a zeta receptor and at least one cancer chemotherapeutic agent; and b) a sustained release formulation. In certain embodiments of methods of inhibiting proliferation or metastasis of a cancer, the medicament includes a peptide ligand of a zeta receptor that may be an enkephalin, a fragment of an enkephalin, or a derivative thereof; or an endorphin, a fragment of an endorphin, or a derivative thereof, or a dynorphin, a fragment of a dynorphin, or a derivative thereof. In yet additional embodiments the medicament includes one or more cancer chemotherapeutic agents.

In still additional embodiments of methods of inhibiting proliferation or metastasis of a cancer the medicament includes both a peptide ligand of a zeta receptor and at least one cancer chemotherapeutic agent. The peptide component may be an enkephalin, a fragment of an enkephalin, or a derivative thereof; or an endorphin, a fragment of an endorphin, or a derivative thereof; or a dynorphin, a fragment of a dynorphin, or a derivative thereof.

The one or more cancer chemotherapeutic agents in the medicament employed in methods of inhibiting proliferation or metastasis of a cancer may be chosen, by way of nonlimiting example, from among asparaginase, hydroxyurea, altretamine, bleomycin, dactinomycin, doxorubicin, etoposide, teniposide, plicamydin, nitrogen mustards such as chlorambucil, cyclophosphamide, isofamide, mechlorethamine, melphalan, uracil mustard; an aziridine such as thiotepa, methanesulphonate esters such as busulfan, nitroso ureas such as carmustine, lomustine, streptozocin; platinum complexes, such as cisplatin and carboplatin; mitomycin, procarbazine, dacarbazine; amsacrine, daunorubicin, adriamycin, idarubicin, mitoxantrone; etoposide; teniposide, folate antagonists such as methotrexate and trimetrexate; pyrimidine antagonists, such as fluorouracil, fluorodeoxyuridine, CB3717, azacytidine, cytarabine, and floxuridine; purine antagonists such as mercaptopurine, 6-thioguanine, fludarabine, pentostatin, vincristine, vinblastine, paclitaxel, estrogens, conjugated estrogens, ethinyl estradiol and diethylstilbesterol, chlortrianisen and idenestrol; progestins such as hydroxyprogesterone caproate, medroxyprogesterone, and megestrol; androgens such as testosterone, testosterone propionate; fluoxymesterone, and methyltestosterone, prednisone, dexamethasone, methylprednisolone, prednisolone, leuprolide acetate, goserelin acetate, tamoxifen, flutamide; mitotane; and aminoglutethimide.

Nonlimiting examples of monoclonal antibody cancer therapeutic agents that may be used in the medicaments employed in methods of inhibiting proliferation or metastasis of the invention include Rituximab (Rituxan), Trastuzumab (Herceptin), Alemtuzumab (Campath), Cetuximab (Erbitux), Bevacizumab (Avastin), Ibritumomab (Zevalin), or Tositumomab, or two or more of them.

The sustained release formulation included in the medicament that is used to contact the tissue may be any of a wide range of formulations. In general, sustained release formulations employed in the compositions, combinations and methods of the present invention are biocompatible, and in many embodiments are biodegradable. Nonlimiting examples of such formulations include gels, hydrogels, formulations applied by spray, bioadhesive formulations, and similar formulations, certain examples of which have been described hereinabove. Thus, by way of nonlimiting example, a sustained release formulation may include a natural polymer, such as a protein, a polysaccharide, a mucopolysaccharide, and the like. A protein component may be an albumin, a fibrinogen or a fibrin or solubilized derivative thereof, any of the several types of collagen in both fibril form or disaggregated, any of the several types of collagen that are atelocollagens, any of the several types of collagen that have been partially degraded in molecular weight, various gelatins derived from the several types of collagen, and the like. Polysaccharides include cellulose, cellulose derivatives such as alkyl celluloses, carboxyalkyl celluloses, glycogen, biocompatible vegetable gums, chitin, chitosan, biocompatible seaweed gums, chondroitin sulfate, hyaluronic acid, and the like.

Further, by way of nonlimiting example, a sustained release formulation may include a synthetic polymer, such as a homopolymers or a copolymer, wherein the copolymer may be a homogeneously copolymerized copolymer or a block copolymer. Components of synthetic polymers may be chosen, by way of nonlimiting example, from among poly(hydroxyacyl)esters; poly(lactone)esters; various polyvinyl derivatives including polyvinylpyrrolidone, polyvinyl alcohol, and the like; polyethyleneoxide and homologous polyalkyleneoxides, both capped and uncapped; polycarbonates; polyanhydrides; polyamines; polyurethanes; polyesteramides; polyorthoesters; polydioxanones; polyacetals, polyketals; polycarbonates; polyorthocarbonates; polyphosphazenes; succinates; poly(malic acid); and poly(amino acids).

A sustained release formulation used in a medicament that is disposed on a tissue may be comprised of a single polymer, or a combination or mixture of more than one polymer. A polymer or copolymer chosen for use is to be biocompatible. In many embodiments the polymer additionally is biodegradable. A polymer used in a medicament may be formulated in an aqueous medium, or a dispersing solvent may include biocompatible nonaqueous solvents or cosolvents according to the properties of the chosen polymer, to permit the composition to be employed as a gel, a hydrogel, a formulation applied by spray, a bioadhesive formulation, or a similar formulation.

The one or more pharmaceutical active agents included in the pharmaceutical composition that is disposed on the tissue, in methods of inhibiting proliferation or metastasis of a cancer, may be incorporated as a solution, a dispersion, a suspension, a comminution, or any similar composition, according to the respective properties of the active agent and the sustained release formulation.

Additionally, sustained release formulations employed to form a medicament disposed on a tissue may be constituted of, or may include as one of several components, particles, beads, microparticles, nanoparticles, and similar formed entities that include a pharmaceutically active agent as a component.

Furthermore, a sustained release composition or medicament disposed on a tissue may contain any of the pharmaceutically active agents disclosed herein, or a precursor thereof that gives rise to the pharmaceutically active agent in situ, conjugated to a polymer component of a sustained release formulation.

Nonlimiting examples of sustained release formulations that may be used in methods of inhibiting proliferation or metastasis of the present invention include those disclosed in patents and patent application publications cited herein, and incorporated by reference herein in their entireties.

In general, in further embodiments of methods of inhibiting proliferation or metastasis of a cancer, release of the peptide ligand, or the one or more cancer chemotherapeutic agents, or both, from the pharmaceutical composition employed in the contacting step occurs in a substantially therapeutically effective amount over a time period ranging from about 1 day to about 13 weeks. Thus the bioavailability may endure 1 day or longer, or about 2 days or longer, or about 4 days or longer, or about 1 week or longer, or about 10 days or longer, or about 2 weeks or longer, or about 20 days or longer, or about 3 weeks or longer, or about 4 weeks or longer, or about 5 weeks or longer, or about 6 weeks or longer, or about 7 weeks or longer, or about 8 weeks or longer, or about 9 weeks or longer, or about 10 weeks or longer, or about 11 weeks or longer, or about 12 weeks or longer, or about 13 weeks or longer, or even longer periods than any of the above.

A medicament disposed on a tissue in methods of inhibiting proliferation or metastasis of a cancer may furthermore be composed of more than one composition, each of which includes one or both of a peptide ligand that binds a zeta receptor and at least one cancer chemotherapeutic agent, and a sustained release formulation that differs from the others. By way of nonlimiting example, a first pharmaceutical composition including one or both of a first peptide ligand that binds a zeta receptor and at least one first cancer chemotherapeutic agent, and a first sustained release formulation, may be combined with a second pharmaceutical composition including one or both of a second peptide ligand that binds a zeta receptor and at least one second cancer chemotherapeutic agent, and a second sustained release formulation. The various component pharmaceutical compositions may include different active agents, and may be characterized by having different sustained release formulations whose release kinetics may be the same, or may that may be similar, or that differ substantially from each other. In general the medicament may be so constituted that a peptide ligand or a cancer chemotherapeutic agent may be released with substantially different kinetics such that one or the other is released over an early time interval, and a second, third, fourth, and so on, agent is released at, or over, a longer time interval or at or over a later time interval. Any of the component compositions in the combined medicament described above may release its pharmaceutical active agent in 1 day or greater, or about 2 days or greater, or about 4 days or greater, or about 1 week or greater, or about 10 days or greater, or about 2 weeks or greater, or about 20 days or greater, or about 3 weeks or greater, or about 4 weeks or greater, or about 5 weeks or greater, or about 6 weeks or greater, or about 7 weeks or greater, or about 8 weeks or greater, or about 9 weeks or greater, or about 10 weeks or greater, or about 11 weeks or greater, or about 12 weeks or greater, or about 13 weeks or greater, or even longer periods than any of the above.

In additional embodiments of methods of inhibiting proliferation or metastasis of a cancer further treatment may include, in addition to the disposing step, systemic administration of a cancer chemotherapeutic agent. Such administration may be any of a variety of parenteral routes, including, by way of nonlimiting example, intravenous, subcutaneous, intramuscular, intraperitoneal, aerosol inhalation, buccal, vaginal, rectal, and the like. Further treatment may be administered on a single occasion, or it may be repeated according to a treatment regimen.

The tissue containing one or more tumor cells harboring a zeta receptor may be a tissue of any mammal, including a human. The inhibition of proliferation or metastasis can generally be provided by any inhibition of growth of the tumor cells as compared to the tumor cells without treatment. The inhibition is preferably at least about 10%, 20%, 30%, 40%, 50%, 60%, 70%, 80%, 90%, 95%, 96%, 97%, 98%, 99%, and ideally 100% inhibition of growth. The inhibition may be achieved by arrest of cell growth, by lysis of the cancer cells or by other means.

Therapeutic Dose

A pharmaceutical composition of the invention is loaded with one or more pharmaceutically active agents in concentrations sufficient to provide a therapeutically effective amount of the agent or agents to cells or tissues in the immediate vicinity of pharmaceutical composition; i.e., as part of a combination of the invention, the pharmaceutical composition provides a therapeutically effective amount of the agent or agents to the tissue with which it is in contact.

A worker of skill in fields such as pharmaceutical science, drug delivery, oncology, and similar fields related to the field of the invention understands the principles and methods of empirically determining effective amounts of the active agent or agents to be loaded into the pharmaceutical compositions of the invention. Factors that a worker of skill considers in arriving at effective pharmaceutical compositions include, by way of nonlimiting example, 1) the pharmaceutical properties of the sustained release formulation employed; 2) the duration of sustained delivery intended for the sustained release formulation; 3) the Kd for binding of an agent to a zeta receptor, or the ED50 (where ED50 designates an effective concentration sufficient to achieve a 50% favorable response in a particular experimental model system) for an agent, where an agent is an enkephalin or derivative thereof, an endorphin or derivative thereof, or a dynorphin or derivative thereof, or a cancer chemotherapeutic agent; 4) the pharmacokinetics of the agent or agents incorporated into the pharmaceutical composition; 5) the amount of the pharmaceutical composition that may be employed in forming the combination of the invention, or in methods of treating a pathology of the invention, or in methods of inhibiting proliferation or metastasis of a cancer of the invention; and related considerations known to the worker of skill.

By way of nonlimiting example, a pharmaceutical composition of the invention may have a loading of at least about 1% by weight of the active agent or agents, or at least about 2% by weight, or at least about 5% by weight, or at least about 8%, or at least about 10% by weight, or at least about 15% by weight, or at least about 20% by weight, or at least about 25% by weight, or at least about 30% by weight, or at least about 35% by weight, or at least about 40% by weight, or at least about 45% by weight, or at least about 50% by weight, or an even higher percent by weight of the active agent or agents. As an alternative way of considering the loading, a pharmaceutical composition of the invention may provide a total cumulative dose over the period of time over which sustained release occurs of at least about 0.1 mg/kg of the active agent or agents, or at least about 0.2 mg/kg, or at least about 0.5 mg/kg, or at least about 1 mg/kg, or at least about 2 mg/kg, or at least about 5 mg/kg, or at least about 10 mg/kg, or at least about 15 mg/kg, or at least about 20 mg/kg, or at least about 25 mg/kg, or at least about 30 mg/kg, or at least about 40 mg/kg, or at least about 50 mg/kg, or at least about 75 mg/kg, or at least about 100 mg/kg, or at least about 150 mg/kg, or at least about 200 mg/kg, or at least about 300 mg/kg, or at least about 400 mg/kg, or at least about 500 mg/kg of the active agent or agents over the entire period of sustained release, or even higher doses per kg of weight of a subject.

Criteria that may be used in establishing effective loadings include various diagnostic procedures such as, by way of nonlimiting example, assessing the level of a characteristic marker of a particular cancer in a biological sample from a subject, diagnostic imaging appropriate for a particular cancer in a subject, pathological examination of biopsy sample from a subject, and related diagnostic procedures known to a worker of skill in fields related to the present invention.

### EXAMPLES

### Example 1

### Inhibiting Recurrence of Disease after Removal of a Solid Tumor

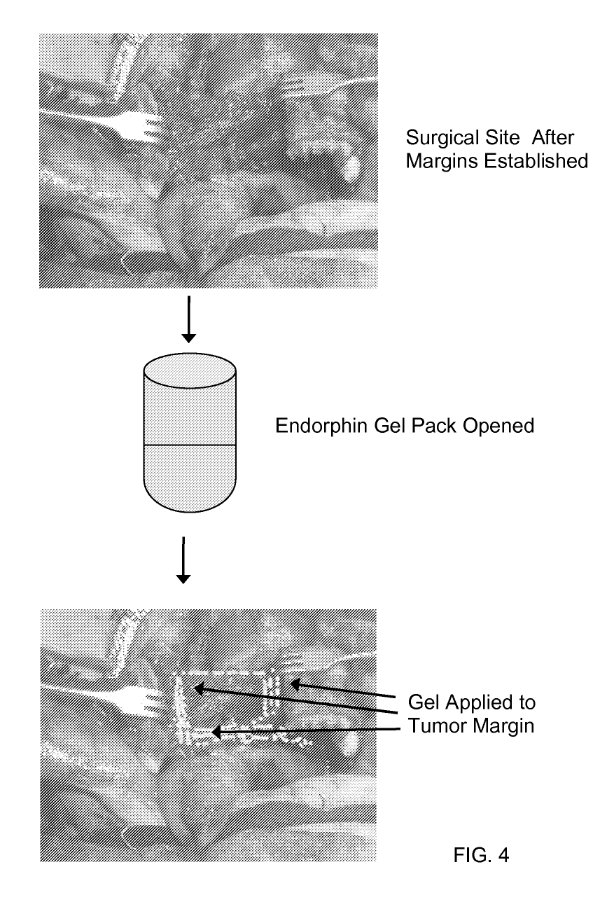

A solid tumor is removed from a subject by surgical resection. An image of internal structures remaining after surgical removal of the tumor is shown in FIG. 4, top. The margins of the cavity are sampled and examined for maximal removal of tumor margin tissue. This procedure ensures minimal residue of tumor cells or precancerous cells remaining at the site of the tumor. An endorphin-containing pharmaceutical composition, designated as an “Endorphin Gel Pack” in FIG. 4, center, is applied to the surgical cavity (see FIG. 4, bottom). The surgical incision in the subject is closed and the subject is allowed to recover. The endorphin contained in the Endorphin Gel Pack is expected to inhibit proliferation or metastasis of the cancer.

### Example 2

### Inhibiting Progression to Cancer of a Precancerous Lesion

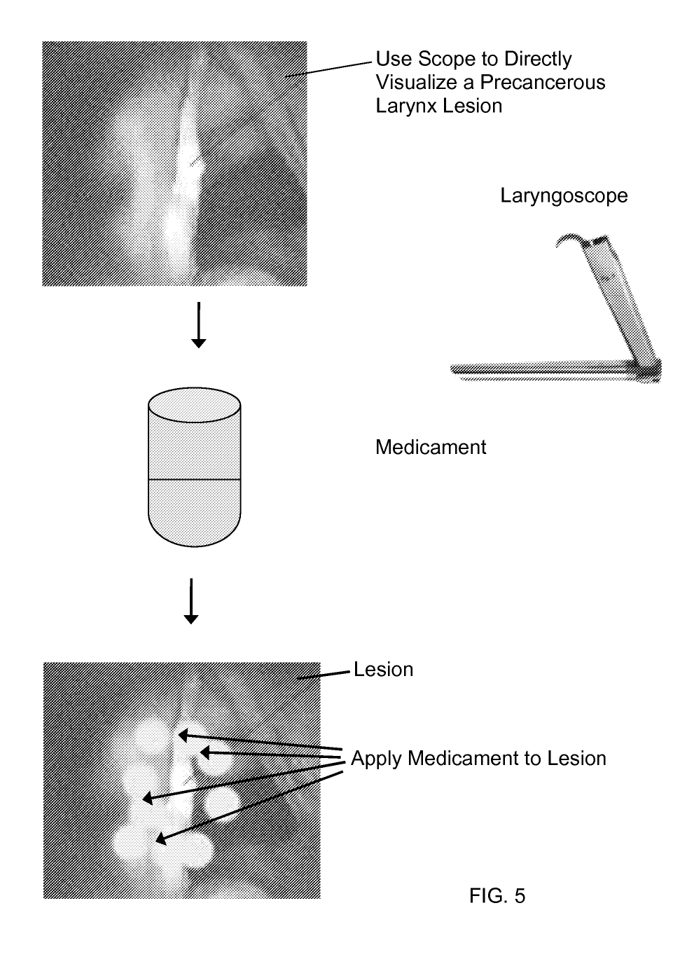

A solid precancerous lesion is identified by direct visual observation in the larynx of a subject, using a laryngoscope or similar suitable implement (see FIG. 5, top). An endorphin-containing pharmaceutical composition, designated as “Medicament” in FIG. 5, center, is applied to the surface of the lesion (see FIG. 5, bottom). The subject is allowed to recover from the procedure. The endorphin contained in the Endorphin Gel Pack is expected to inhibit progression of the lesion to develop cancer.

I claim:

1. A pharmaceutical composition comprising
a) one or both of
i) a peptide ligand that binds a zeta receptor and
ii) at least one cancer chemotherapeutic agent; and
b) a sustained release formulation suitable for topical application to a tissue.

2. The composition described in claim 1 wherein the composition comprises the peptide ligand.

3. The composition described in claim 1 wherein the composition comprises at least one cancer chemotherapeutic agent.

4. The composition described in claim 1 wherein the peptide ligand comprises an enkephalin, an endorphin, a dynorphin, or a derivative or fragment of any of them.

5. The composition described in claim 1 wherein the composition comprises both a peptide ligand that binds a zeta receptor and at least one cancer chemotherapeutic agent.

6. The composition described in claim 1 wherein the sustained release formulation comprises a gel, a hydrogel, a bioadhesive, a crosslinked gel, a microsphere, a microparticle, or a nanoparticle, or a combination of any of them.

7. The composition described in claim 1 wherein the composition comprises a sustained release formulation comprising a polymeric carrier conjugated to one or both of
a) a peptide ligand that binds a zeta receptor and
b) at least one cancer chemotherapeutic agent.

8. A method of treating a pathology that responds to a peptide ligand that binds a zeta receptor comprising disposing a pharmaceutical composition upon at least a portion of a tissue suspected of comprising one or more cells exhibiting the pathology wherein the one or more cells harbor a zeta receptor, wherein the pharmaceutical composition comprises
a) one or both of
i) a peptide ligand that binds a zeta receptor and
ii) at least one cancer chemotherapeutic agent, in amounts effective to treat the pathology; and
b) a sustained release formulation suitable for topical application to a tissue.

9. The method described in claim 8 wherein the pathology is a cancer.

10. The method described in claim 8 wherein the one or more cells is a cancer cell or a precancerous cell, or both.

11. The method described in claim 8 wherein the one or more cells are at a margin of a tumor wherein the margin is exposed by removal of a preponderance of the tumor.

12. The method described in claim 8 wherein the pharmaceutical composition comprises the peptide ligand.

13. The method described in claim 8 wherein the pharmaceutical composition comprises at least one cancer chemotherapeutic agent.

14. The method described in claim 8 wherein the peptide ligand comprises an enkephalin, an endorphin, a dynorphin, or a derivative or fragment of any of them.

15. The method described in claim 8 wherein the pharmaceutical composition comprises both a peptide ligand that binds a zeta receptor and at least one cancer chemotherapeutic agent.

16. The method described in claim 8 wherein release of the peptide ligand, or the one or more cancer chemotherapeutic agents, or both, comprised therein from the pharmaceutical composition occurs in a substantially therapeutically effective amount over a time period ranging from about 1 day to about 13 weeks.

17. The method described in claim 8 wherein the composition comprises a sustained release formulation comprising a polymeric carrier conjugated to one or both of
a) a peptide ligand that binds a zeta receptor and
b) at least one cancer chemotherapeutic agent.

18. A method of inhibiting proliferation or metastasis of a cancer in a subject comprising the steps of
a) surgically excising a tumor characterizing the cancer from the subject;
b) ensuring maximal removal of a tumor margin from the tumor site; and
c) disposing a pharmaceutical composition upon at least a portion of a tissue remaining at a tumor margin wherein the tissue is suspected of comprising one or more tumor cells harboring a zeta receptor, the pharmaceutical composition comprising
i) one or both of
a′) a peptide ligand that binds a zeta receptor and
b′) at least one cancer chemotherapeutic agent, in amounts effective to treat the cancer; and
ii) a sustained release formulation suitable for topical application to a tissue;
thereby inhibiting proliferation or metastasis of the cancer.

19. The method described in claim 18 wherein the peptide ligand comprises an enkephalin or a derivative thereof.

20. The method described in claim 18 wherein the peptide ligand comprises an enkephalin, an endorphin, a dynorphin, or a derivative or fragment of any of them.

21. The method described in claim 18 wherein the composition comprises a sustained release formulation comprising a polymeric carrier conjugated to one or both of
a) a peptide ligand that binds a zeta receptor and
b) at least one cancer chemotherapeutic agent.
